(19) United States
(12) Patent Application Publication
(10) Pub. No.: US 2022/0370487 A1
(43) Pub. Date: Nov. 24, 2022

(54) COMPOSITIONS TARGETING SODIUM CHANNEL 1.6

(71) Applicant: Q-STATE BIOSCIENCES, INC., Cambridge, MA (US)

(72) Inventors: Christina Ambrosi, Cambridge, MA (US); Luis Williams, Cambridge, MA (US); Caitlin Lewarch, Cambridge, MA (US); David Gerber, Cambridge, MA (US); Owen McManus, Belmont, MA (US); Sudhir Agrawal, Cambridge, MA (US); Graham T. Dempsey, Sudbury, MA (US)

(21) Appl. No.: 17/740,485

(22) Filed: May 10, 2022

Related U.S. Application Data
(60) Provisional application No. 63/186,342, filed on May 10, 2021.

(51) Int. Cl. A61K 31/7088

(52) U.S. Cl. CPC A61K 31/7088

(57) ABSTRACT

The invention provides therapeutic compositions that include an antisense oligonucleotide (ASO) complementary to an identified target on a Nav channel mRNA. The ASO hybridizes to its target RNA and forms a duplex that recruits RNase H to degrade the RNA, thereby downregulating Nav channel synthesis, which inhibits the neuron's ability to contribute to certain conditions such as epilepsy and pain perception. The ASO binds to one of the specific identified targets, and may be provided as a gapmer that includes a central DNA segment flanked by modified RNA wings.

### TECHNICAL FIELD

The disclosure relates to compositions that inhibit activity of voltage-gated sodium channel 1.6.

### BACKGROUND

Sodium channels are membrane proteins that function as channels that pass sodium ions (Na+) across the plasma membrane of a cell. Sodium channels may be ligand-gated or voltage gated, and voltage gated channels for Na+ are referred to as NaV channels. In cells such as neurons and cardiomyocytes, NaV channels are responsible for the rising phase of action potentials. NaV channels have three states: resting, active and inactive, distinguished by structural conformation of the protein.

Because cardiac activity, brain function, and physical sensation require cardiomyocytes and neurons, the function of which depends on sodium channels, sodium channels have been looked to as targets for the treatment of cardiac arrythmias, neurological conditions, and pain. In one example, procainamide has been used to treat atrial fibrillation and complex tachycardias. In another example, the small molecule Funapide is in development as an analgesic. The anticonvulsants phenytoin and carbamazepine are used to treat epilepsy and are understood to function as sodium channel blockers. The list of reported potential therapeutic targets for sodium channel blockers includes chronic pain, migraines, epilepsy, cardiovascular diseases, psychiatric disorders, and even cancer. See Li, 2019, Voltage-gated sodium channels and blockers: an overview and where will they go?, Curr Med Sci 39(6):867-873, incorporated by reference. Unfortunately, the usefulness of small molecule sodium channel blockers may be limited by state-dependency, binding kinetics, and access to the target, which require that the drug finds and interacts with the target protein in a particular conformational state.

### SUMMARY

The invention provides compositions that inhibit or knock-down expression of the Nav1.6 protein. Compositions of the invention are potentially useful to treat or diagnose conditions in which sodium channel function is implicated such as epilepsy, cardiovascular diseases, pain, or psychiatric disorders, including in some embodiments developmental and epileptic encephalopathy, 13 (DEE13). Compositions of the invention are also useful as prophylactic treatments. There are nine families of voltage-gated sodium channels in humans, named Nav1.1 through Nav1.9. Of those nine proteins, Nav1.6, encoded by the SCN8A gene (12q13.13), regulates the initiation of action potentials and participates in nerve conduction. Nav1.6 is abundantly expressed in the brain and is important to neural function. Compositions of the disclosure may be used to knock down expression of Nav1.6 for the treatment of conditions in which neural activity and sodium channel function play a role. In particular, compositions of the disclosure may find benefit as therapeutic treatments for conditions such as epilepsy, pain or conditions involving neuronal hyperexcitability.

In some embodiments, compositions of the disclosure may be useful as state-independent anti-epileptic drugs or anticonvulsants. Exome sequencing has shown a link between SCN8A and epilepsy. It is thought that mutations in SCN8A may cause epilepsy and associated seizures. Specifically, gain-of-function SCN8A mutations may cause hyperexcitability and impaired channel inactivation. In fact, seizures are treated with small-molecule anti-epileptic drugs that function by blocking sodium channels. See Zaman, 2019, A single-center SCN8A-related epilepsy cohort: clinical, genetic, and physiologic characterization, Ann Clin Trans Neurol 6(8):1445-1455 and Boerma, 2016, Remarkable phenytoin sensitivity in 4 children with SCN8A-related epilepsy: a molecular neuropharmacological approach, Neurotherapeutics 13(1):192-197, both incorporated by reference. Compositions of the disclosure may be used to knock down expression of Nav1.6 in a state-independent manner for the treatment of, for example, epilepsy including for the treatment of seizures. Such an approach may be used to treat DEE13, a developmental and epileptic encephalopathy resulting from mutations in the SCN8A gene. Such an approach is also applicable to treat Dravet Syndrome and other forms of epilepsy, including but not limited to forms resulting from mechanisms of excessive excitation and inhibition (E/I) balance.

In certain embodiments, compositions of the disclosure may be useful as analgesics for the treatment of pain including, for example, chronic pain conditions such as cancer pain or arthritis. The compositions include short nucleic acids, or oligonucleotides, that prevent the synthesis of proteins involved in neural activity. For example, some neurons operate as “pain-sensing” nerves, or nociceptors. Those pain-sensing neurons have proteins that function as voltage-gated sodium channels. When stimulation of the nerve endings exceeds a threshold voltage (V), the nociceptor neurons conduct sodium ions (Na+) across the cell membrane, which can cause the neuron to depolarize in a regenerative fashion leading to “firing” of propagating electrical signals that underlie the sensation of pain. Compositions of the disclosure may be used to knock down expression of Nav1.6 in a state-independent manner for the treatment of pain.

The invention provides compositions that inhibit or knock down expression of the Nav1.6 protein. Compositions of the invention include oligonucleotides that bind to the messenger RNA (mRNA) or precursor mRNA (pre-mRNA) used in making the Nav1.6 sodium channel proteins. The invention includes the identification of numerous specific validated targets within those RNAs. The oligonucleotides are substantially or entirely antisense to the targets and are described as antisense oligonucleotides (ASOs). The oligonucleotides prevent those proteins from being made, which decreases the sensitivity or activity of cells expressing those sodium channels. Because Nav1.6 expression is knocked down in those cells, those cells do not contribute to conditions such as chronic pain, migraines, epilepsy, cardiovascular diseases, or certain psychiatric disorders. Thus, compositions of the disclosure provide for state-independent therapeutic treatments for a variety of conditions that offer alternatives to the limitations of small-molecule channel blockers where usefulness may be limited by binding kinetics or conformational state-dependency, or access to the target ion channel.

Oligonucleotides described by the disclosure are designed to hybridize to certain targets in the RNAs used in synthesis of Nav1.6 protein. Binding of the oligonucleotides prevents protein synthesis and downregulates expression of the NaV channel. Specifically, the oligonucleotides have a sequence that is substantially or entirely complementary to one of the identified targets on a NaV channel precursor mRNA (pre-mRNA) or mRNA. That is, the oligonucleotide is antisense to the identified target. When the antisense oligonucleotide (ASO) hybridizes to its target RNA, they form a double-stranded ASO:RNA duplex that recruits an enzyme (RNase H) that degrades a portion of the double-stranded duplex. Degrading the ASO:RNA duplex depletes the cell (e.g., neuron) of NaV channel mRNA, which decreases the amount of NaV channel synthesized by the cell. Downregulating NaV channel expression interferes with the ability of the neuron to contribute to epileptic activity or the sensation of pain. Thus, when a composition that includes oligonucleotides that are antisense to the identified targets in Nav1.6 pre-mRNA or mRNA is administered to a patient, that patient may have a diminished risk of seizures or experience of pain.

For some embodiments, the invention targets a region of the transcript identified here as a hotspot to beneficially target with ASOs of the disclosure. Empirical data emerged from the results presented here, showing that certain ASOs targeting targets within about the first 3700 bases of the SCN8A transcript are highly efficacious at knockdown by the desired amounts. A number of the ASOs disclosed herein are specific to that 3700 base region and data show that those ASOs give unexpectedly good results.

Another insight of the invention is that certain ASOs knock down SCN8A by very beneficial amounts and not by 100%. Without being bound by any mechanism of action, it may be theorized that complete knockdown (or knockout) is adverse, but that the beneficial effects may be had by using ASOs that exhibit a saturating profile, or plateau, that does not reach 100% knockdown, i.e., 0% relative normalized expression. In fact, certain ASOs of the disclosure provide that benefit as a result of their sequence and it is not strictly a dose-dependent effect. Those ASOs plateau at some % knockdown less than 100 as dose concentration increases. Certain embodiments disclosed here knock down expression of SCN8A by an amount between about 50 and 80%, e.g., by about 60%, even at high concentrations or doses.

In certain aspects, the disclosure provides a composition that includes an oligonucleotide that hybridizes to a pre-mRNA or mRNA encoding a sodium channel protein along a segment of that RNA that is at least 75% complementary to one of SEQ ID NOs: 1-156 to thereby prevent translation of the RNA into the sodium channel protein. The oligonucleotide may hybridize to, and knock down expression of, Nav1.6 pre-mRNA or mRNA. Preferably, a sequence of bases in the oligonucleotide has at least 80% identity to one of SEQ ID NOs: 1-144. For example, the sequence of bases in the oligonucleotide may be at least 90% or 95% identical to one of SEQ ID NOs: 1-144, and the oligonucleotide may hybridize to, and induce RNase H cleavage of, either Nav1.6 pre-mRNA or mRNA. The composition may include a plurality of therapeutic oligonucleotides each having a base sequence at least 80, 90, 95, or 100% identical to one of SEQ ID NOs: 1-156. Certain preferred ASOs include eleven that are complementary to targets within exons of the human SCN8A gene and five that are complementary to targets within introns of the human SCN8A gene. The eleven preferred ASOs that are complementary to targets within exons of the human SCN8A gene include those referred to by Ref. Nos.: 14-016 (SEQ ID NO: 16); 14-041 (SEQ ID NO: 41); 14-044 (SEQ ID NO: 44); 14-045 (SEQ ID NO: 45); 14-100 (SEQ ID NO: 100), 14-117 (SEQ ID NO: 117), 14-124 (SEQ ID NO: 124), 14-125 (SEQ ID NO: 125), 14-126 (SEQ ID NO: 126), 14-128 (SEQ ID NO: 128), 14-129 (SEQ ID NO: 129), 14-130 (SEQ ID NO: 130), 14-133 (SEQ ID NO: 133), 14-134 (SEQ ID NO: 134), 14-135 (SEQ ID NO: 135), 14-138 (SEQ ID NO: 138), 14-139 (SEQ ID NO: 139), 14-142 (SEQ ID NO: 142), 14-143 SEQ ID NO: 143), and 14-144 (SEQ ID NO: 144). Certain most preferred embodiments may include SEQ ID NO: 016, 041, 044, 045, 117, 124, 135, or 144.

The five preferred ASOs that are complementary to targets within introns of the human SCN8A gene include those referred to by Ref. Nos.: 14-100 (SEQ ID NO: 100); 14-101 (SEQ ID NO: 101); 14-102 (SEQ ID NO: 102); 14-103 (SEQ ID NO: 103); and 14-104 (SEQ ID NO: 104).

Some embodiments includes ASOs that target a region of the first 3700 bases of SCN8A transcript such as, for example one of ASO 14-001 (SEQ ID NO: 1); 14-002 (SEQ ID NO: 2); 14-003 (SEQ ID NO: 3); 14-004 (SEQ ID NO: 4); 14-005 (SEQ ID NO: 5); 14-006 (SEQ ID NO: 6); 14-007 (SEQ ID NO: 7); 14-008 (SEQ ID NO: 8); 14-009 (SEQ ID NO: 9); 14-010 (SEQ ID NO: 10); 14-011 (SEQ ID NO: 11); 14-012 (SEQ ID NO: 12); 14-013. (SEQ ID NO: 13); 14-014 (SEQ ID NO: 14); 14-015 (SEQ ID NO: 15); 14-016 (SEQ ID NO: 16); 14-041 (SEQ ID NO: 41); 14-042 (SEQ ID NO: 42); 14-043 (SEQ ID NO: 43); 14-044 (SEQ ID NO: 44); 14-045 (SEQ ID NO: 45); 14-046 (SEQ ID NO: 46); 14-047 (SEQ ID NO: 47); 14-048 (SEQ ID NO: 48); 14-049 (SEQ ID NO: 49); 14-050 (SEQ ID NO: 50); 14-051 (SEQ ID NO: 51); 14-115 (SEQ ID NO: 115); 14-116 (SEQ ID NO: 116); 14-117 (SEQ ID NO: 117); 14-118 (SEQ ID NO: 118); 14-119 (SEQ ID NO: 119); 14-120 (SEQ ID NO: 120); 14-121 (SEQ ID NO: 121); 14-122 (SEQ ID NO: 122); 14-123 (SEQ ID NO: 123); 14-124 (SEQ ID NO: 124); 14-125 (SEQ ID NO: 125); 14-126 (SEQ ID NO: 126); 14-127 (SEQ ID NO: 127); 14-128 (SEQ ID NO: 128); 14-129 (SEQ ID NO: 129); 14-130 (SEQ ID NO: 130); 14-131 (SEQ ID NO: 131); 14-132 (SEQ ID NO: 132); 14-133 (SEQ ID NO: 133); 14-134 (SEQ ID NO: 134); 14-135 (SEQ ID NO: 135); 14-136 (SEQ ID NO: 136); 14-137 (SEQ ID NO: 137); 14-138 (SEQ ID NO: 138); 14-139 (SEQ ID NO: 139); 14-140 (SEQ ID NO: 140); 14-141 (SEQ ID NO: 141); 14-142 (SEQ ID NO: 142); 14-143 (SEQ ID NO: 143); 14-144 (SEQ ID NO: 144); 14-145 (SEQ ID NO: 145); 14-146 (SEQ ID NO: 146); 14-147 (SEQ ID NO: 16); 14-148 (SEQ ID NO: 16); 14-149 (SEQ ID NO: 41); 14-150 (SEQ ID NO: 41); 14-151 (SEQ ID NO: 44); 14-152 (SEQ ID NO: 44); 14-153 (SEQ ID NO: 45); 14-154 (SEQ ID NO: 45); 14-155 (SEQ ID NO: 117); 14-156 (SEQ ID NO: 117); 14-157 (SEQ ID NO: 124); 14-158 (SEQ ID NO: 124); 14-159 (SEQ ID NO: 126); 14-160 (SEQ ID NO: 126); 14-161 (SEQ ID NO: 129); 14-162 (SEQ ID NO: 129); 14-163 (SEQ ID NO: 133); 14-164 (SEQ ID NO: 133); 14-165 (SEQ ID NO: 135); 14-166 (SEQ ID NO: 135); 14-167 (SEQ ID NO: 138); 14-168 (SEQ ID NO: 138); 14-169 (SEQ ID NO: 139); 14-170 (SEQ ID NO: 139); 14-171 (SEQ ID NO: 142); 14-172 (SEQ ID NO: 142); 14-173 (SEQ ID NO: 143); 14-174 (SEQ ID NO: 143); 14-175 (SEQ ID NO: 144); or 14-176 (SEQ ID NO: 144). Particular embodiments may use one of ASOs 14-147 (SEQ ID NO: 16); 14-148 (SEQ ID NO: 16); 14-149 (SEQ ID NO: 41); 14-150 (SEQ ID NO: 41); 14-151 (SEQ ID NO: 44); 14-152 (SEQ ID NO: 44); 14-153 (SEQ ID NO: 45); 14-154 (SEQ ID NO: 45); 14-155 (SEQ ID NO: 117); 14-156 (SEQ ID NO: 117); 14-157 (SEQ ID NO: 124); 14-158 (SEQ ID NO: 124); 14-159 (SEQ ID NO: 126); 14-160 (SEQ ID NO: 126); 14-161 (SEQ ID NO: 129); 14-162 (SEQ ID NO: 129); 14-163 (SEQ ID NO: 133); 14-164 (SEQ ID NO: 133); 14-165 (SEQ ID NO: 135); 14-166 (SEQ ID NO: 135); 14-167 (SEQ ID NO: 138); 14-168 (SEQ ID NO: 138); 14-169 (SEQ ID NO: 139); 14-170 (SEQ ID NO: 139); 14-171 (SEQ ID NO: 142); 14-172 (SEQ ID NO: 142); 14-173 (SEQ ID NO: 143); 14-174 (SEQ ID NO: 143); 14-175 (SEQ ID NO: 144); or 14-176 (SEQ ID NO: 144).

Certain preferred embodiments may use ASOs for which SCN8A knockdown plateaus beneath 100% as dose increases. Those embodiments may use one of ASOs 14-135, 14-165, 14-166, 14-144, 14-175, and 14-175, which are here shown to not achieve knockdown of 100% and instead to plateau between about 70 and 80% even at high dose concentration, which is desired.

Certain embodiments use a gapmer ASO with a sequence given by at least one of SEQ ID NO: 16; SEQ ID NO: 41; SEQ ID NO: 44; SEQ ID NO: 45; SEQ ID NO: 117; SEQ ID NO: 124; SEQ ID NO: 126; SEQ ID NO: 129; SEQ ID NO: 133; SEQ ID NO: 135; SEQ ID NO: 138; SEQ ID NO: 139; SEQ ID NO: 142; SEQ ID NO: 143; or SEQ ID NO: 144, in which the gapmer has a 12 base DNA central segment flanked by 2′-MOE RNA wings, in which each wing has one or two phosphodiester linkages with remaining inter-base linkages being phosphorothioate, with all cytosine bases having a 5-methyl modification. Certain most preferred embodiments (for a knockdown plateau achieving expression between about 50 and 90% normalized to untreated) use 14-165, 14-166, 14-175, or 14-176. Here, 14-165 is SEQ ID NO: 135 in a gapmer with a 12 base DNA central segment flanked by 2′-MOE RNA wings, in which the 2d, 3rd, and 18th inter-base linkages are phosphodiester, with remaining inter-base linkages being phosphorothioate, and all cytosine bases having a 5-methyl modification. 14-166 is SEQ ID NO: 135 in a gapmer with a 12 base DNA central segment flanked by 2′-MOE RNA wings, in which 2d, 3rd, 4th, and 18th inter-base linkages are phosphodiester with remaining inter-base linkages being phosphorothioate, and all cytosine bases having a 5-methyl modification. 14-175 is SEQ ID NO: 144 in a gapmer with a 12 base DNA central segment flanked by 2′-MOE RNA wings, in which 2d, 3rd, and 18th inter-base linkages are phosphodiester with remaining inter-base linkages being phosphorothioate, and all cytosine bases having a 5-methyl modification. 14-176 is SEQ ID NO: 144 in a gapmer with a 12 base DNA central segment flanked by 2′-MOE RNA wings, in which 2d, 3rd, 4th, and 18th inter-base linkages are phosphodiester with remaining inter-base linkages being phosphorothioate, and all cytosine bases having a 5-methyl modification.

Therapeutic oligonucleotides of the disclosure may have a gapmer structure that includes a central DNA segment flanked by modified RNA wings. Such a therapeutic oligonucleotide may include two wings flanking a central region of DNA bases (e.g., about 8 to 14 DNA bases, e.g., 12). Preferably at least one end of the oligonucleotide comprises modified RNA bases, e.g., any number or any combination of 2′-O-methoxyethyl RNA (“2′-MOE”) and/or 2′-O-methyl RNA (“2′-O-Me”). Bases may be modified. For example, a percentage or all cytosines may be methylated (e.g., 5-methyl cytosines) in the 5′ RNA wing, the central DNA region, the 3′ RNA wing, or all three.

The therapeutic oligonucleotide may be provided in a solution or carrier formulated for delivery such as by injection. The oligonucleotide may be of any suitable length, e.g., at least about 15 bases, preferably between about 15 and 25 bases. The oligonucleotide may have phosphorothioate bonds in the backbone. In preferred embodiments, the oligonucleotide has a base sequence that has been screened and determined not to meet a threshold match for any long, non-coding RNA or other off-target sequences or transcripts in humans. The oligonucleotide may have a base sequence with 0 mismatches to a homologous segment in a non-human primate genome and no more than about 5 mismatches in a homologous segment in a rodent genome.

When the composition is delivered to SK-N-AS neuroblastoma cells in vitro, the cells exhibit a dose-dependent knockdown of Nav1.6. The oligonucleotide may be a gapmer having a base sequence with at least a 90% match to one of SEQ ID NO: 1-156 (preferably at least a 90% match to one of 14-016 (SEQ ID NO: 16), 14-041 (SEQ ID NO: 41), 14-044 (SEQ ID NO: 44), 14-045 (SEQ ID NO: 450, 14-100 (SEQ ID NO: 100), 14-117 (SEQ ID NO: 117), 14-124 (SEQ ID NO: 124), 14-125 (SEQ ID NO: 125), 14-126 (SEQ ID NO: 126), 14-128 (SEQ ID NO: 128), 14-129 (SEQ ID NO: 129), 14-130 (SEQ ID NO: 130), 14-133 (SEQ ID NO: 133), 14-134 (SEQ ID NO: 134), 14-135 (SEQ ID NO: 135), 14-138 (SEQ ID NO: 138), 14-139 (SEQ ID NO: 139), 14-142 (SEQ ID NO: 142), 14-143 SEQ ID NO: 143), and 14-144 (SEQ ID NO: 144), with bases linked by phosphorothioate linkages. The linkages may be all phosphorothioate or a mixture of phosphorothioate and phosphodiester bonds. The oligonucleotide may further have a central region between about 8 and about 14 DNA bases flanked by a 5′ wing and a 3′ wing, the 5′ wing and the 3′ wing each comprising a number of, e.g., a few, consecutive 2′ modified RNA bases. Preferably, the oligonucleotide has a base sequence matching one of SEQ ID NO: 1-156, with bases linked by phosphorothioate linkages, and a structure having central DNA bases flanked by a 5′ wing and a 3′ wing. The numbers of RNA bases in the wings and DNA bases in the central segment may be 4-12-4, 5-10-5, 5-9-5, 4-11-4, or a similar suitable pattern. The 5′ wing and the 3′ wing may each include several 2′-MOE RNA bases. For example, the oligonucleotide may have 4 consecutive 2′-MOE RNA bases in each wing with a central 12 DNA bases (a “4-12-4” structure), with phosphorothioate linkages throughout the central DNA segment and a mixture of phosphorothioate and phosphodiester bonds in the wings.

In combination embodiments, the invention provides compositions that include a plurality of copies of a plurality of distinct therapeutic gapmers, each according to the descriptions above, in a suitable formulation or carrier.

In some aspects, the disclosure provides a method of treating epilepsy. The method includes administering to a subject with epilepsy a composition as described herein to thereby knockdown expression of a SCN8A gene. The epilepsy being treated my be DEE13, Dravet syndrome, or any epilepsy involving a pathogenic mechanism of excessive E/I balance.

Aspects of the disclosure provide a use of an antisense oligonucleotide (ASO) for the manufacture of a medicament for treating a condition such as epilepsy or pain in a patient. In the use, the ASO has at least about 75% identity with one of SEQ ID NOs: 1-156, and more preferably at least 90% identity, e.g., 95% or 100% identity. Preferred embodiments use an ASO that is between about 15 and 25 bases in length, preferably between about 18 and 22, or between about 19 and 21 (inclusive). In general, reference to “an ASO” includes numerous copies of substantially identical molecules. Accordingly, “an ASO” may be any number, e.g., hundreds of thousands, or millions, of copies of the indicated ASO. In preferred embodiments, the ASO is 20 bases in length and has the sequence of one of SEQ ID NOs: 1-156 and is used in the manufacture of a medicament for the treatment of a condition. The ASO may be provided in any suitable format such as, for example, lyophilized in a tube or in solution in a tube, such as a microcentrifuge tube or a test tube. Preferred embodiments of the use target Nav1.6. One or more (e.g., two, three, four, or five, or more) ASOs may be used in manufacture of the medicament. The one or more ASOs may hybridize to a target in a Nav1.6 pre-mRNA or mRNA. In certain embodiments of the use, a sequence of bases in the ASO is at least 90% identical to one of SEQ ID NOs: 1-156. In certain preferred embodiments, a sequence of bases in the ASO is at least about 95% identical to, or preferably matches, one of 14-016 (SEQ ID NO: 16), 14-041 (SEQ ID NO: 41), 14-044 (SEQ ID NO: 44), 14-045 (SEQ ID NO: 450, 14-100 (SEQ ID NO: 100), 14-117 (SEQ ID NO: 117), 14-124 (SEQ ID NO: 124), 14-125 (SEQ ID NO: 125), 14-126 (SEQ ID NO: 126), 14-128 (SEQ ID NO: 128), 14-129 (SEQ ID NO: 129), 14-130 (SEQ ID NO: 130), 14-133 (SEQ ID NO: 133), 14-134 (SEQ ID NO: 134), 14-135 (SEQ ID NO: 135), 14-138 (SEQ ID NO: 138), 14-139 (SEQ ID NO: 139), 14-142 (SEQ ID NO: 142), 14-143 SEQ ID NO: 143), and 14-144 (SEQ ID NO: 144). In embodiments of the use, an ASO may have a gapmer structure with a central DNA segment flanked by RNA wings, e.g., a central region of 12 DNA bases with 4 modified RNA bases on both sides of the central region. Each modified RNA base may be 2′-MOE. Preferably a backbone of the ASO has a plurality of phosphorothioate bonds, e.g. several, many, most, or all of the sugar linkages may be phosphorothioate in the use embodiments (balance phosphodiester). The medicament may include the ASO in a form suitable for mixing into a formulation suitable for introduction by injection, an infusion, or a pump. For example, the ASO (thousands or millions or more of copies of one ASO) may be lyophilized in a tube or in solution at a known molality or concentration. The ASO may be dissolved or diluted into a pharmaceutically acceptable composition in which a carrier, such as a solvent and/or excipient, includes the ASO and may be loaded in an IV bag, syringe, or pump. The medicament may be made using more than one ASO, e.g., any combination of 2, 3, 4, or 5, or more.

### BRIEF DESCRIPTION OF THE DRAWINGS

FIG. 1 shows a composition for treating epilepsy.

FIG. 2 shows an oligonucleotide with a gapmer structure.

FIG. 3 shows a 2′-O-Methoxyethyl (MOE) modified ribose sugar.

FIG. 4 shows a phosphorothioate bond in a segment of DNA.

FIG. 5 shows expression of SCN8A after treatment with ASOs of SEQ ID Nos: 1-74 (first 20 bars in each panel shows treatment with 1 ASO, 21^{st} bar is NT siRNA; see key).

FIG. 6 shows expression of SCN8A after treatment with ASOs of SEQ ID Nos: 75-114.

FIG. 7 gives results of a reproducibility analysis for ASOs of the disclosure in different replicate cultures.

FIG. 8 shows dose response under treatment with ASOs of SEQ ID Nos: 1-4, 6, 8, 11, 16, 41, 44, 45, and 100-104.

FIG. 9 shows percent knockdown resulting from treatment with SCN8A exon-targeting ASOs tiling the full transcript.

FIG. 10 shows percent knockdown resulting from treatment with SCN8A intron-targeting ASOs.

FIG. 11 shows percent knockdown resulting from treatment with SCN8A ASOs all targeting the first 3700 bases of the transcript.

FIG. 12 shows percent knockdown for other sodium channels (encoded by SCN2A, SCN3A, and SCN9A) resulting from treatment with SCN8A exon-targeting ASOs tiling the full transcript.

FIG. 13 shows percent knockdown for other sodium channels (encoded by SCN2A, SCN3A, and SCN9A) resulting from treatment with SCN8A intron-targeting ASOs.

FIG. 14 shows percent knockdown for other sodium channels (encoded by SCN2A, SCN3A, and SCN9A) resulting from treatment with optimized SCN8A exon-targeting ASOs.

FIG. 15 shows dose-response data for certain ASOs in SK-N-AS neuroblastoma cells.

FIG. 16 shows expanded dose-response data for certain ASOs in SK-N-AS neuroblastoma cells.

FIG. 17 shows the dose-response percent knockdown for the lead all-PS backbone candidates targeting SCN8A exons.

FIG. 18 shows the dose-response percent knockdown for PO-modified daughter leads for human clinical candidates.

FIG. 19 shows knockdown of SCN8A transcript in human NGN2 stem cell-derived neurons.

FIG. 20 shows knockdown of SCN8A transcript in human primary neurons using lead candidates.

FIG. 21 shows knockdown of Scn8a transcript in mouse primary cortical neurons.

FIG. 22 shows Scn8a transcript knockdown in rat primary hippocampal neurons.

FIG. 23 shows evidence of a plateau in SCN8A transcript knockdown in human NGN2 stem cell-derived neurons.

### DETAILED DESCRIPTION

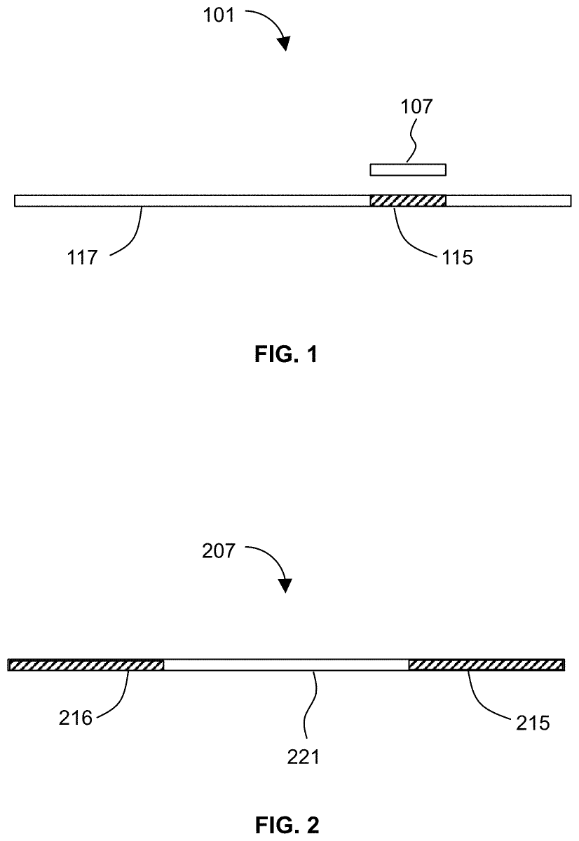

FIG. 1 shows a composition 101 for treating epilepsy or pain. The composition 101 includes an oligonucleotide 107 that hybridizes to a target segment 115 in an mRNA 117 or a pre-mRNA. The mRNA 117 encodes a sodium channel protein. The segment 115 of the mRNA 117 that includes the target is at least about 75% complementary to one of SEQ ID NOs: 1-156.

Hybridization of the oligonucleotide 107 to the segment 115 of the mRNA 117 prevents translation of the mRNA into the sodium channel protein (e.g., by recruiting RNase H leading to digestion of the double-stranded oligonucleotide 107/RNA 117 complex or by interfering with protein translation). The oligonucleotide 107 may hybridize to, and knock down expression of, Nav1.6 pre-mRNA or mRNA. Preferably, a sequence of bases in the oligonucleotide has at least 80% identity to one of SEQ ID NOs: 1-156, and more preferably at least 90% identity, e.g., at least 95% identity.

In certain embodiments, a sequence of bases in the oligonucleotide is at least 90% identical to one of SEQ ID NOs: 1-156, wherein the oligonucleotide can hybridize to, and induce RNase H cleavage of, Nav1.6 pre-mRNA or mRNA.

The oligonucleotide 107 hybridizes to the segment 115 in the RNA 117 because the oligonucleotide 107 is substantially or entirely antisense to the target segment 115 of the mRNA 117. In that sense, the composition includes an antisense oligonucleotide. Compositions 101 include ASOs that bind to target RNA with base pair complementarity and exert various effects, based on the ASO chemical structure and design. Various mechanisms, commonly employed in preclinical models of neurological disease and human clinical trial development, may be employed. Those mechanisms include RNA target degradation via recruitment of the RNase H enzyme.

Preferred embodiments of the disclosure include ASOs that hybridize to voltage gated sodium channel (NaV channel) pre-mRNA or mRNA and recruit the RNase H enzyme. The RNase H enzyme cleaves the NaV channel RNA, which downregulates expression of the NaV channel protein. Thus, oligonucleotide 107 of the disclosure addresses NaV channels as targets for therapy for conditions such as epilepsy including e.g., DEE13, Dravet syndrome and forms of epilepsy with pathogenic mechanism involving excessive E/I balance ratio as non-limiting examples. The disclosure builds on the insights that clinical and preclinical data support the use of small molecule NaV blockers for therapy, e.g., as anticonvulsants or analgesics. For example, dibucaine, methyl benzetheonium chloride, darifenacin hydrobromide, and dimethisoquin hydrochloride have all been investigated for their inhibitory activity against Nav1.6 channels. See Atkin, 2018, A comprehensive approach to identifying repurposed drugs to treat SCN8A epilepsy, Epilepsia 59(4):802-813, incorporated by reference. Similarly, sodium channels have been implicated in pain. Compositions that include anti-NaV ASOs may be administered to a subject to treat conditions such as epilepsy or pain. It may be found that anti-NaV ASOs offer benefits over other approaches such as small molecule blockers because anti-NaV ASOs may be state-independent and subtype selective.

Thus, the disclosure provides a use of an antisense oligonucleotide (ASO) for the manufacture of a medicament for treating a condition in a patient. In the use, the ASO has at least about 75% identity with one of SEQ ID NOs: 1-156, and more preferably at least 90% identity, e.g., 95% or greater identity. Preferred embodiments use an ASO that is between about 15 and 25 bases in length, preferably between about 18 and 22 (inclusive). In general, reference to “an ASO” includes numerous copies of substantially identical molecules. Accordingly, “an ASO” may be more than hundreds of thousands or millions of copies of the defined ASO. In preferred embodiments, the ASO is 20 bases in length and has the sequence of one of SEQ ID NOs: 1-156 and is used in the manufacture of a medicament. The ASO may be provided in any suitable format such as, for example, lyophilized in a tube or in solution in a tube, such as a microcentrifuge tube or a test tube. Preferred embodiments of the use target Nav1.6. One or more (e.g., a combination of two, three, four, or five, or more) ASOs may be used in manufacture of the medicament. The one or more ASOs may hybridize to a target in a Nav1.6 RNA. In certain embodiments of the use, a sequence of bases in the ASO is at least 90%, preferably at least 95% or 100%, identical to one of SEQ ID NOs: 1-156. E.g., a sequence of bases in the ASO may be at least 90%, 95% or 100% identical to one of the preferred sequences.

In embodiments of the use, an ASO may have a gapmer structure with a central DNA segment flanked by RNA wings, e.g., a central region of 12 DNA bases with 4 modified RNA bases on both sides of the central region, i.e., a 4-12-4 structure. The structure may be 5-10-5, or 4-9-4, or 4-10-4-, or 5-9-5, or similar. Each modified RNA base may be 2′-MOE RNA, 2′-O-Me RNA, or other suitable sugar. Preferably a backbone of the ASO has a plurality of phosphorothioate bonds, either exclusively or also including phosphodiester linkages, e.g., most or all of the sugar linkages may be phosphorothioate in the use embodiments. The medicament may be formulated for delivery, e.g., via injection. Accordingly, the ASO may initially be in a form suitable for mixing into a formulation suitable for introduction into an IV bag, syringe, or intrathecal pump. For example, the ASO (thousands or millions or more of copies of one ASO) may be lyophilized in a tube or in solution at a known molality of concentration. The ASO may be dissolved or diluted into a pharmaceutically acceptable composition in which a carrier, such as a solvent or excipient, includes the ASO and may be loaded in an IV bag, syringe, or intrathecal pump. The medicament may be made using more than one ASO, e.g., any combination of 2, 3, 4, or 5, or more.

Any ASO(s) described in the use embodiment may be included in a composition of the disclosure. Preferred embodiments of compositions of the disclosure include one or a plurality of therapeutic oligonucleotides each having a base sequence at least 90% identical to one of SEQ ID NOs: 1-156, wherein each of the therapeutic oligonucleotides has a gapmer structure that comprises a central DNA segment flanked by modified RNA wings, wherein the plurality of therapeutic oligonucleotides are provided in a solution or carrier formulated for intrathecal injection.

FIG. 2 shows an oligonucleotide 207 with a gapmer structure. The oligonucleotide 207 includes two wings (first wing 215 and second wing 216) flanking a central region 221 of about 12 DNA bases. In preferred embodiments, the wings 215, 216 are all or predominantly RNA bases whereas the central region 221 is all or predominantly DNA bases. Preferably, the wings are all RNA bases (modified or unmodified) and the central region is all DNA bases. In some embodiments, each wing consists of 4 RNA bases, all or most of which are modified RNA bases, e.g., in which each modified RNA base is selected from the group consisting of 2′-O-methoxyethyl RNA and 2′-O-methyl RNA. A modified RNA base may include a substitution on a 2′ hydroxyl group of a ribose sugar.

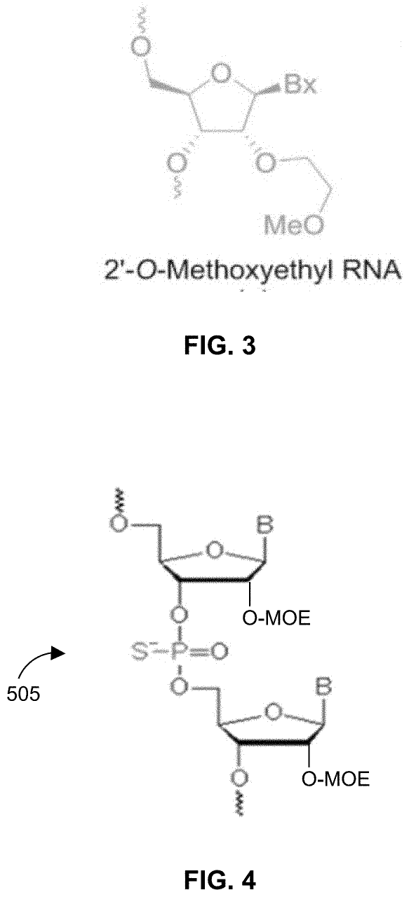

FIG. 3 shows a 2′-O-Methoxyethyl (“2′-MOE”) modified sugar that may be included in an RNA base.

The oligonucleotide 207 preferably includes at least about 15 bases, and may include between about 15 and about 25 bases. In some embodiments, the oligonucleotide 207 has a backbone comprising a plurality of phosphorothioate bonds.

FIG. 4 shows a phosphorothioate bond 505 within the backbone of a segment of DNA, such as the central region 221 of the oligonucleotide 207. The oligonucleotide 207 may include one or any number of the phosphorothioate bonds 505. For example, every backbone linkage within the oligonucleotide 207 may be phosphorothioate, or most, or about half may be.

The composition 101 may be formulated for delivery. Accordingly, the oligonucleotide 107 may initially be in a form suitable for mixing into a formulation suitable for introduction into a syringe, bag, or injection pump. For example, the oligonucleotide 107 (thousands or millions or more of copies of one oligonucleotide 107) may be lyophilized in a tube or in solution at a known molality of concentration. The oligonucleotide 107 may be dissolved or diluted into a pharmaceutically acceptable composition in which a carrier, such as a solvent or excipient, includes the oligonucleotide 107 and may be loaded in an IV bag, syringe, or intrathecal pump. As described, the composition 101 includes at least one oligonucleotide 107 with a sequence that is defined by comparison to one of SEQ ID NO: 1-156. Thus, compositions of the disclosure are defined and illustrated by the identified targets.

Specifically, the oligonucleotide 107 hybridizes to an mRNA encoding a sodium channel protein along a segment of the mRNA that is at least about 75% complementary to one of SEQ ID NOs: 1-156 to thereby prevent translation of the mRNA into the sodium channel protein. This is accomplished where the oligonucleotide has at least about 75% identity to one of SEQ ID Nos: 1-156, preferably at least about 90% or 95% identity. In certain embodiments, the oligonucleotide has the sequence of one of SEQ ID Nos: 1-156, although one of skill in the art will understand that oligonucleotides with 90% or preferably 95% identity to a complementary target will still tend to hybridize in a sequence-specific manner to the target. Forming a double stranded structure is energetically favorable enough through Watson-Crick base pairing and base stacking that the double stranded structure can tolerate approximately about 1 mismatched base pair every ten or so. Accordingly, under moderately stringent physiological conditions in a cell, 95% identity should be effective, especially where an oligonucleotide has a gapmer structure with at least a few modified RNA bases or phosphorothioate backbone linkages to protect the oligonucleotide from enzymatic degradation.

In fact, a feature and benefit of compositions of the disclosure is that the targets (SEQ ID Nos: 1-156) have been screened to rule out sequences for which the complement is present in molecules other than sodium channel transcripts. For example, the sequences have been screened against databases of RNA transcripts including long, non-coding RNA (lncRNA), and initial sequences that matched non-target sequences were excluded. Thus, ASOs with sequences of SEQ ID Nos. 1-156 when administered to a patient should have a minimized chance of hybridizing to non-target sequences. Accordingly, in preferred embodiments, the oligonucleotide 107 has a base sequence that has been screened and determined to not meet a threshold match for any off-target coding or long, non-coding RNA in humans. A composition or use that meets the criteria stated above should not bind to off-target material such as lncRNA in vivo, as the included sequences have been screened against a database of lncRNA. Sequences of the disclosure have been screened for target specificity. Preferably, the oligonucleotide 107 has a base sequence with 0 mismatches to a homologous segment in a non-human primate genome and no more than about 5 mismatches in a homologous segment in a rodent genome.

When the composition is delivered to cells in vitro, the cells exhibit a dose-dependent knockdown of Nav1.6.

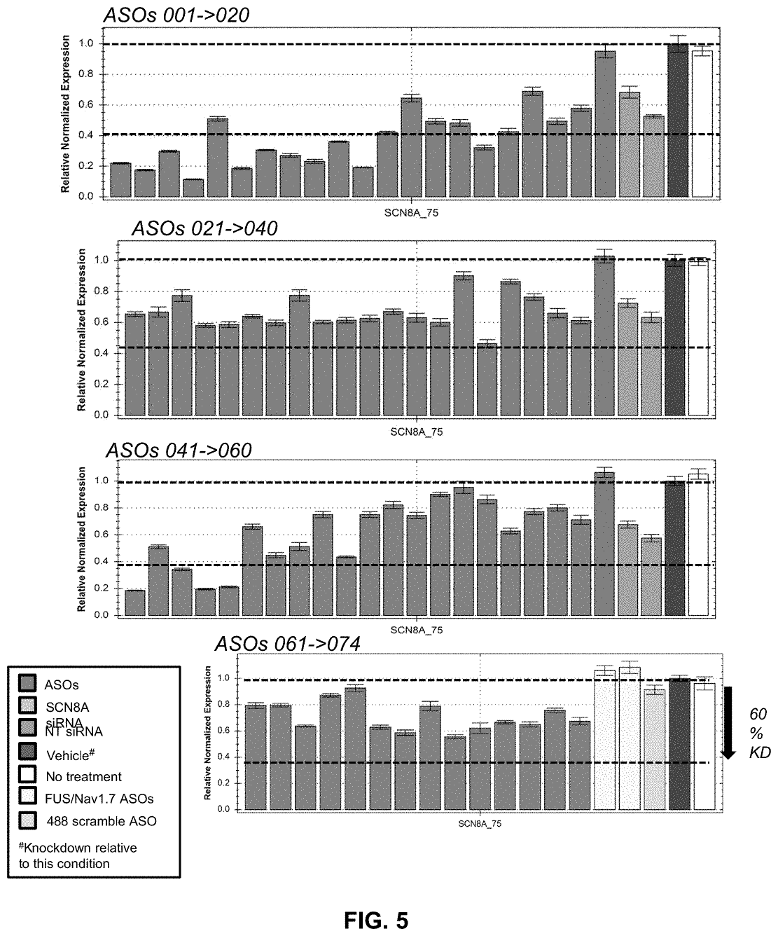

FIG. 5 shows the relative normalized expression (on the y-axis) of SCN8A for exonic ASOs of SEQ ID Nos. 1-74 delivered at 100 nM to SK-N-AS cells. In each panel, the first 20 bars corresponds to one respective ASO. The 21^{st} and subsequent bars corresponds to one of the controls shown in the key. The ASO bars are in numerical order by SEQ ID No, i.e., so that in the third panel, labeled “ASOs 041->060”, the first bar is a result from an ASO of SEQ ID NO: 41 and fourth bar is a result from an ASO of SEQ ID NO: 44. The bars show that all of the ASOs exhibited some knockdown compared to vehicle, and ASOs of SEQ ID NOs:1-4, 6-11, 16, 41, and 43-45 exhibited very good knockdown of about 60%.

In all cases, an ASO is identified by the final 3 digits, such that ASO-001 is 001 is 14-001 is QS-Ts14-ASO-001. Those are equivalent labels referring to the same thing. The shorter version is used in some place for formatting.

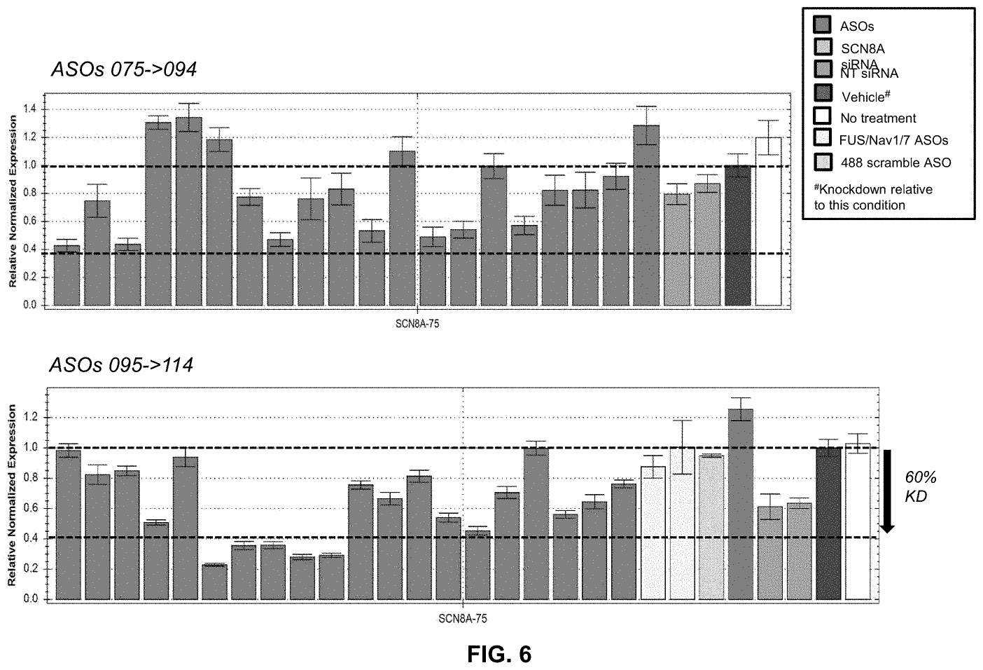

FIG. 6 shows the relative normalized expression (on the y-axis) of SCN8A for intronic ASOs of SEQ ID Nos. 75-114 delivered at 100 nM to SK-N-AS cells. In the figures, for comparison, expression is measured for cells un-treated, treated with small, interfering RNA, vehicle (no ASO), off-target ASOs (e.g., against NaV 1.7), and “scrambled” ASOs. The bars show that most of the ASOs exhibited some expression knockdown compared to vehicle and that ASOs of SEQ ID NOs: 75, 77, 82, 85, 87, 88, 90, 98, 100-104, and 209 exhibited very good knockdown e.g., about ≥60%.

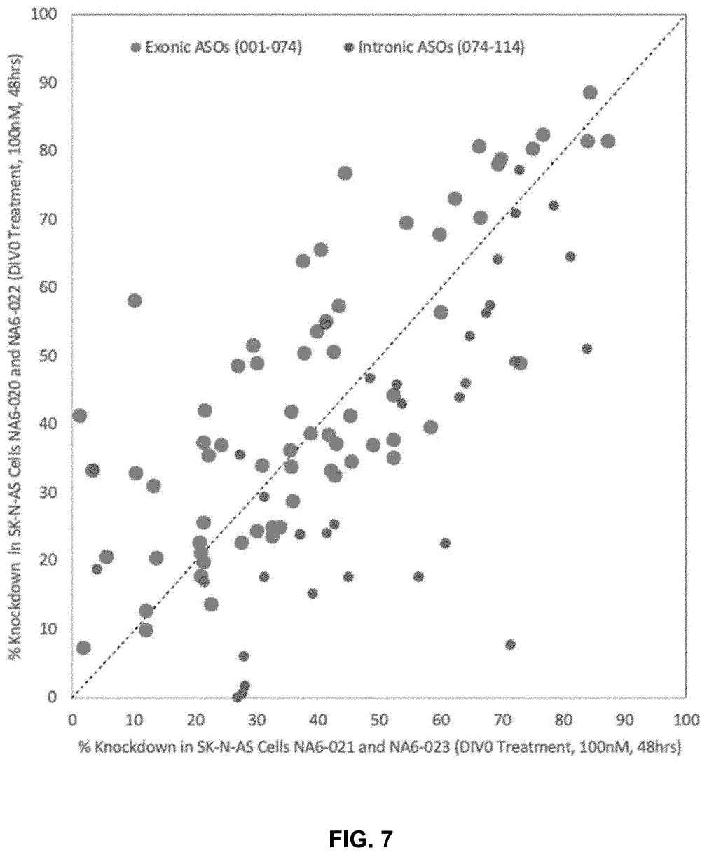

FIG. 7 gives results of a combined reproducibility analysis for ASOs of SEQ ID Nos. 1-74 targeting SCN8A exons and ASOs of SEQ ID Nos. 75-114 targeting SCN8A introns. The scatterplot shows knockdown across two replicate experiments along with the prioritization of 16 ASOs (11 exonic and 5 intronic) which show >60% transcript knockdown at a single dose. The experiments involved single-dose ASO screening (at 100 nM with transfection) in SK-N-AS neuroblastoma cells. The axes are different replicate tests. The exonic ASOs of SEQ ID Nos: 1-3, 4, 6, 8, 11, 16, 41, 44, and 45 and the intronic ASOs of SEQ ID Nos: 100-104 are the 16 that were prioritized and selected for dose-dependency testing.

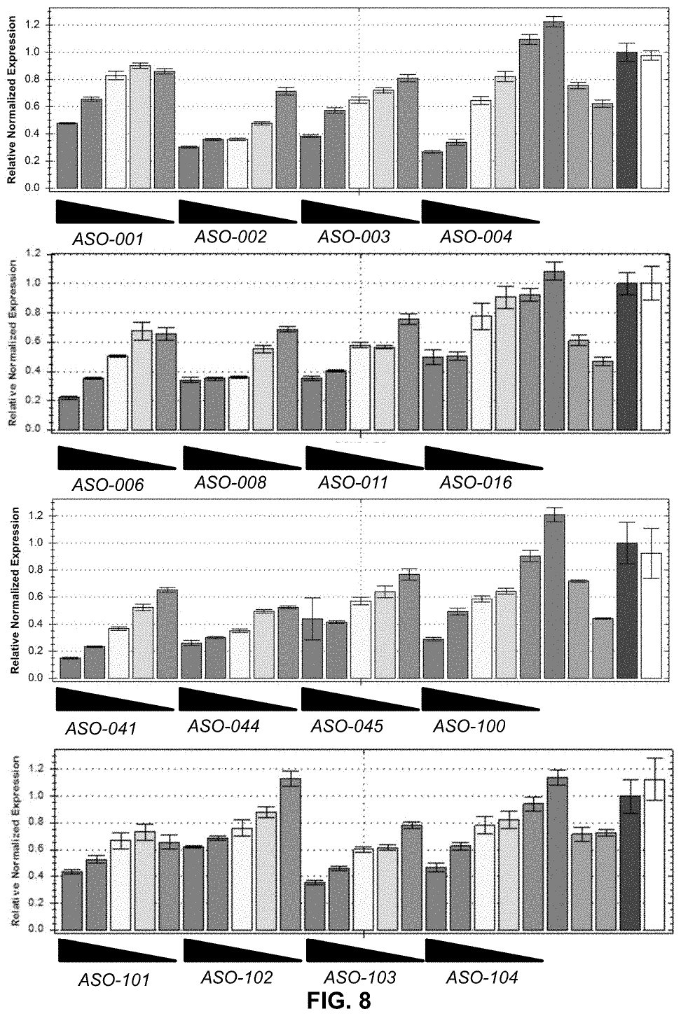

FIG. 8 shows dose-dependent effects of the selected ASOs. The graph gives SCN8A relative normalized expression quantities at day in vitro 2 (DIV 2) 48 hrs post-ASO treatment with the selected ASOs of SEQ ID Nos: 1-3, 4, 6, 8, 11, 16, 41, 44, 45, and 100-104, each at 5 concentrations in increments from 6.25 nM to 100 nM in SK-N-AS cells, at round 2. The dose-response was completed with a 16-fold concentration range (100, 50, 25, 12.5, and 6.25 nM). The ASOs tested for dose-dependency were made according to embodiments of the disclosure (20 bases, 12 base DNA central region flanked by RNA wings with 2′-MOE RNA with 5-methyl cytosine and phosphorothioate linkages throughout the ASO). The right most 5 bars show the expression levels when siRNA was used, a scramble ASO was used, vehicle alone (no ASO), and no treatment. All 16 ASOs decreased Nav1.6 expression, relative to controls, in a dose-dependent manner. The graph shows that a composition 101 of the disclosure exhibits dose-dependent knockdown of Nav1.6.

Because nucleic acid hybridization has some tolerance for mismatches, it may be found that an oligonucleotide 107 with a base sequence that is at least a 90% match to one of SEQ ID NO: 1-156, with bases linked only by phosphorothioate linkages, and in which the oligonucleotide 107 has a central segment of DNA bases flanked by a 5′ wing and a 3′ wing (e.g., a 4-12-4 structure in which the 5′ wing and the 3′ wing each comprise four consecutive 2′ modified RNA bases flanking 12 DNA bases, or a 5-10-5 structure, or similar) exhibits dose-dependent knockdown according to the pattern shown in the chart. In some embodiments, the oligonucleotide 107 specifically has a base sequence matching one of SEQ ID NO: 1-156, with bases linked by phosphorothioate linkages (optionally with some phosphodiester linkages in the wings), in which the oligonucleotide 107 has a central 12 DNA bases flanked by a 5′ wing and a 3′ wing, and in which the 5′ wing and the 3′ wing each include four consecutive 2′-MOE RNA bases.

Because these compositions are effective at knocking down expression of sodium channels, the compositions of the disclosure may be used to treat conditions in which neuronal activity plays a critical role, e.g., conditions such as epilepsy or pain. In such conditions, the electrophysiology of neurons is implicated and in some such conditions, the activity of neurons may be characterized by a condition-specific phenotype, which may be detected by action potential shape or spike patterns shown in neural activity. Compositions of the disclosure may restore a condition-specific phenotype to a healthy phenotype and that restorative effect may be demonstrable in vitro via electrophysiology assays, e.g., on neurons in vitro. The effects of compounds on neurons may be demonstrated using optogenetic assays with in vitro neurons. For example, in vitro neurons may include optogenetic constructs that provide neural activation under optical stimulus (e.g., a modified algal channelrhodopsin that causes the neuron to fire in response to light) and optical reporters of neural activity (modified archaerhodopsins that emit light in proportion to neuronal membrane voltage and yield signals of neuronal activity). The in vitro neurons may be assayed in a fluorescence microscopy instrument. See US Pub 2021/0138039, incorporated by reference. Any suitable optogenetic constructs, optogenetic microscope, or pain mediator compositions may be used. For example, suitable optogenetic constructs include those described in U.S. Pat. No. 9,594,075, incorporated by reference. Suitable optogenetic microscopes include those described in U.S. Pat. No. 10,288,863, incorporated by reference. Suitable pain mediator compositions include those described in WO 2018/165577, incorporated by reference.

In one example of testing a composition for analgesic properties, in vitro DRG assays may involve measuring light from an optogenetic neural sample alone, under increasing optical stimulation. This gives a baseline reading of neural excitability. Then, the neural sample is stimulated with an irritant (e.g., a pain mediator composition comprising a mixture of cytokines, proteases, pH, necrosis factors, or other factors that may be found in vivo at the site of a painful tumor). Light is measured from the neural sample under treatment with that irritant. Finally, the neural sample is treated with a composition of the disclosure. It may be found that, where the irritant measured excitability moves away from the measured baseline, the oligonucleotides 107 will tend to restore measured excitability towards the baseline.

An oligonucleotide of the disclosure, such as a gapmer, ASO, or therapeutic oligonucleotide 107 in a composition 101 may have a sequence defined with reference to one of the sequences set forth in Table 1. For example, an oligonucleotide of the disclosure may have a sequence that is at least about 75%, 80%, 90%, 95%, or perfectly identical to one of SEQ ID NO. 1-156 as set forth in Table 1. The top preferred embodiments against SCN8A include 14-016 (SEQ ID NO: 16); 14-041 (SEQ ID NO: 41); 14-044 (SEQ ID NO: 44); and 14-045 (SEQ ID NO: 45); 14-100 (SEQ ID NO: 100), 14-117 (SEQ ID NO: 117), 14-124 (SEQ ID NO: 124), 14-125 (SEQ ID NO: 125), 14-126 (SEQ ID NO: 126), 14-128 (SEQ ID NO: 128), 14-129 (SEQ ID NO: 129), 14-130 (SEQ ID NO: 130), 14-133 (SEQ ID NO: 133), 14-134 (SEQ ID NO: 134), 14-135 (SEQ ID NO: 135), 14-138 (SEQ ID NO: 138), 14-139 (SEQ ID NO: 139), 14-142 (SEQ ID NO: 142), 14-143 SEQ ID NO: 143), and 14-144 (SEQ ID NO: 144). The data show that compositions of the disclosure show robust and significant knockdown activity (>70%) of Nav1.6 in a dose dependent manner.

### Base Sequences for the ASOs

TABLE 1
Sequences for therapeutic oligonucleotides. Asterisk (*) indicates
ASO that hybridizes within first 3700 bases of SCN8A transcript.
E = exonic; I = intronic; M = exonic with mouse homology.

| ID | SEQ | Sequence | Target |
|---|---|---|---|
| Qs-Tg14-ASO-001* | (SEQ ID NO: 1) | GAAGAGAGTTTTCCCTCTGT | E |
| Qs-Tg14-ASO-002* | (SEQ ID NO: 2) | GGTATCCCTCTGGGCATTGC | E |
| Qs-Tg14-ASO-003* | (SEQ ID NO: 3) | GGGTTCCTTCCTGCTTTCAT | E |
| Qs-Tg14-ASO-004* | (SEQ ID NO: 4) | AGCCTCTTTTTGTTCTGCCT | E |
| Qs-Tg14-ASO-005* | (SEQ ID NO: 5) | GCAGCCTGTGCCTCTTCCTG | E |
| Qs-Tg14-ASO-006* | (SEQ ID NO: 6) | AGGCCTTCCTTCTCATGCCA | E |
| Qs-Tg14-ASO-007* | (SEQ ID NO: 7) | GCACTTTCTCTGAGACTCTT | E |
| Qs-Tg14-ASO-008* | (SEQ ID NO: 8) | GGCACTTTCTCTGAGACTCT | E |
| Qs-Tg14-ASO-009* | (SEQ ID NO: 9) | CCAGTGTCAGGTTGCCCAGG | E |
| Qs-Tg14-ASO-010* | (SEQ ID NO: 10) | GGGAGTTCACAGTCCTGGTT | E |
| Qs-Tg14-ASO-011* | (SEQ ID NO: 11) | GGCCTGCCACTTCCATGCAG | E |
| Qs-Tg14-ASO-012* | (SEQ ID NO: 12) | GGTCCAGGCCACACCCTTCT | E |
| Qs-Tg14-ASO-013* | (SEQ ID NO: 13) | TCCTCTGTGTTGAGGTTCTC | E |
| Qs-Tg14-ASO-014* | (SEQ ID NO: 14) | GGCTGTTCCACAGGGACCTC | E |
| Qs-Tg14-ASO-015* | (SEQ ID NO: 15) | GACCTGGCAGCACTTGAACC | E |
| Qs-Tg14-ASO-016* | (SEQ ID NO: 16) | GTTGACCTGGCAGCACTTGA | E |
| Qs-Tg14-ASO-017 | (SEQ ID NO: 17) | TGTTGACCTGGCAGCACTTG | E |
| Qs-Tg14-ASO-018 | (SEQ ID NO: 18) | GACTTGCCTAGCCCTTCCTC | E |
| Qs-Tg14-ASO-019 | (SEQ ID NO: 19) | GATGAGACACACCAGCAGCA | E |
| Qs-Tg14-ASO-020 | (SEQ ID NO: 20) | TCTCTGTATTGTTCCCCTCC | E |
| Qs-Tg14-ASO-021 | (SEQ ID NO: 21) | GCCAGGTATCCTGCCCCAAC | E |
| Qs-Tg14-ASO-022 | (SEQ ID NO: 22) | ATGAAGATGTCCTGACCTCC | E |
| Qs-Tg14-ASO-023 | (SEQ ID NO: 23) | GTGGCTTCTTTGAGCCCAGC | E |
| Qs-Tg14-ASO-024 | (SEQ ID NO: 24) | ATGTTCTCCATCTGCTTGCT | E |
| Qs-Tg14-ASO-025 | (SEQ ID NO: 25) | GCAGGCCATCCCAACCAGCT | E |
| Qs-Tg14-ASO-026 | (SEQ ID NO: 26) | GGGTCTGCACTTTCCTCTGT | E |
| Qs-Tg14-ASO-027 | (SEQ ID NO: 27) | GGTGTGTGCCTCCATTCTCC | E |
| Qs-Tg14-ASO-028 | (SEQ ID NO: 28) | CCCTTCTTCCTTCCTCTGCC | E |
| Qs-Tg14-ASO-029 | (SEQ ID NO: 29) | GCAAGTCCTCCTCAGCCTGA | E |
| Qs-Tg14-ASO-030 | (SEQ ID NO: 30) | GGCCTGAGCAAGTCCTCCTC | E |
| Qs-Tg14-ASO-031 | (SEQ ID NO: 31) | GCTGGGTCCTGTCCAAAGGC | E |
| Qs-Tg14-ASO-032 | (SEQ ID NO: 32) | GTTGGTTGTTTCCCTACCCA | E |
| Qs-Tg14-ASO-033 | (SEQ ID NO: 33) | GTCTCCCCCATGTACTGGAC | E |
| Qs-Tg14-ASO-034 | (SEQ ID NO: 34) | GGGAAGCCCTGCTCAGTGGA | E |
| Qs-Tg14-ASO-035 | (SEQ ID NO: 35) | TACCCTCACCCACCTCTGTG | E |
| Qs-Tg14-ASO-036 | (SEQ ID NO: 36) | GCTCCATCTCTACCTCAGGT | E |
| Qs-Tg14-ASO-037 | (SEQ ID NO: 37) | GCTCCCCCAGAAGCCACACT | E |
| Qs-Tg14-ASO-038 | (SEQ ID NO: 38) | CCCCCTCTAGTGTATGGTTC | E |
| Qs-Tg14-ASO-039 | (SEQ ID NO: 39) | TCCTAGCAGTTAGCAGGGTC | E |
| Qs-Tg14-ASO-040 | (SEQ ID NO: 40) | GTCTGTCTGTCTGGTTTGGG | E |
| Qs-Tg14-ASO-041* | (SEQ ID NO: 41) | GGTGGTTTCTTGAGCTTGCT | E |
| Qs-Tg14-ASO-042* | (SEQ ID NO: 42) | GTTTGGCTTGGGCTTGCTGT | E |
| Qs-Tg14-ASO-043* | (SEQ ID NO: 43) | TGAAGAGAGTTTTCCCTCTG | E |
| Qs-Tg14-ASO-044* | (SEQ ID NO: 44) | GCCATCTATGCAGAAACCTC | E |
| Qs-Tg14-ASO-045* | (SEQ ID NO: 45) | GCCTTTGGTGCCATTTTCAA | E |
| Qs-Tg14-ASO-046* | (SEQ ID NO: 46) | CAGCCTGTGCCTCTTCCTGT | E |
| Qs-Tg14-ASO-047* | (SEQ ID NO: 47) | GCCTTCCTTCTCATGCCATC | E |
| Qs-Tg14-ASO-048* | (SEQ ID NO: 48) | GAAGATGCTGCTCTTGCTGT | E |
| Qs-Tg14-ASO-049* | (SEQ ID NO: 49) | ATTTCCACCTCAGTTGTAGC | E |
| Qs-Tg14-ASO-050* | (SEQ ID NO: 50) | TGGTGCTCCATTGCCATAAA | E |
| Qs-Tg14-ASO-051* | (SEQ ID NO: 51) | TGCCACTTCCATGCAGTCCC | E |
| Qs-Tg14-ASO-052 | (SEQ ID NO: 52) | TGAGCAACATCTCCAGGATG | E |
| Qs-Tg14-ASO-053 | (SEQ ID NO: 53) | GCCATAGGCTGTCCACTTGA | E |
| Qs-Tg14-ASO-054 | (SEQ ID NO: 54) | ATGAGACACACCAGCAGCAC | E |
| Qs-Tg14-ASO-055 | (SEQ ID NO: 55) | AGAAGGGCCAGGTATCCTGC | E |
| Qs-Tg14-ASO-056 | (SEQ ID NO: 56) | CATGAAGATGTCCTGACCTC | E |
| Qs-Tg14-ASO-057 | (SEQ ID NO: 57) | GCTTCTTTGAGCCCAGCTTT | E |
| Qs-Tg14-ASO-058 | (SEQ ID NO: 58) | GCCAGGAACATTCCCACAAT | E |
| Qs-Tg14-ASO-059 | (SEQ ID NO: 59) | GCAGCAGGCCATCCCAACCA | E |
| Qs-Tg14-ASO-060 | (SEQ ID NO: 60) | GCACTTTCCTCTGTGGCTAC | E |
| Qs-Tg14-ASO-061 | (SEQ ID NO: 61) | GCTCACCATTGGCAGATCCA | E |
| Qs-Tg14-ASO-062 | (SEQ ID NO: 62) | GTGTGTGCCTCCATTCTCCA | E |
| Qs-Tg14-ASO-063 | (SEQ ID NO: 63) | CTTGGATTCTCTGACCTCTT | E |
| Qs-Tg14-ASO-064 | (SEQ ID NO: 64) | CAGTAACCACCTGCCCAAGC | E |
| Qs-Tg14-ASO-065 | (SEQ ID NO: 65) | GAAGCCCATGTGGAGAAGAT | E |
| Qs-Tg14-ASO-066 | (SEQ ID NO: 66) | TGGTTGTTTCCCTACCCAGA | E |
| Qs-Tg14-ASO-067 | (SEQ ID NO: 67) | GCCCTAGGATTTGGTTTTAC | E |
| Qs-Tg14-ASO-068 | (SEQ ID NO: 68) | CCCTAGAGATAACTGGTGCT | E |
| Qs-Tg14-ASO-069 | (SEQ ID NO: 69) | GGCTCCATCTCTACCTCAGG | E |
| Qs-Tg14-ASO-070 | (SEQ ID NO: 70) | CTGCAGCATCTTGGCTTTGA | E |
| Qs-Tg14-ASO-071 | (SEQ ID NO: 71) | AAGTGCTTCTACTGCAAGGC | E |
| Qs-Tg14-ASO-072 | (SEQ ID NO: 72) | CCTAGCAGTTAGCAGGGTCA | E |
| Qs-Tg14-ASO-073 | (SEQ ID NO: 73) | GCCAGAAGCCATTTCCTATT | E |
| Qs-Tg14-ASO-074 | (SEQ ID NO: 74) | GCCCATCTCAAGACCTAAGA | E |
| Qs-Tg14-ASO-075 | (SEQ ID NO: 75) | GTGCTACAGGTAAGCCAGGC | I |
| Qs-Tg14-ASO-076 | (SEQ ID NO: 76) | TGCTATCAGACAGAGGCTGC | I |
| Qs-Tg14-ASO-077 | (SEQ ID NO: 77) | CTGGCAGAGGGAACATGGCT | I |
| Qs-Tg14-ASO-078 | (SEQ ID NO: 78) | ACAGAGGTTCCAGAGTGTTA | I |
| Qs-Tg14-ASO-079 | (SEQ ID NO: 79) | AGCTATATTCCCTCTACCAA | I |
| Qs-Tg14-ASO-080 | (SEQ ID NO: 80) | CTGGTCTTTGGTTTGAGGAA | I |
| Qs-Tg14-ASO-081 | (SEQ ID NO: 81) | GGGACTGGGCTGAGAACAAC | I |
| Qs-Tg14-ASO-082 | (SEQ ID NO: 82) | ACTCTGTTATATACCTGCAG | I |
| Qs-Tg14-ASO-083 | (SEQ ID NO: 83) | TTAATGTGATGAGGCAGCTT | I |
| Qs-Tg14-ASO-084 | (SEQ ID NO: 84) | CTGGAAGGAAAGGTGGTTGA | I |
| Qs-Tg14-ASO-085 | (SEQ ID NO: 85) | AGTTTCTTCTACCCTCCCCC | I |
| Qs-Tg14-ASO-086 | (SEQ ID NO: 86) | GCACTCTCCCCCTTTACCCC | I |
| Qs-Tg14-ASO-087 | (SEQ ID NO: 87) | GCCCTCCCTCTGTCTATCCC | I |
| Qs-Tg14-ASO-088 | (SEQ ID NO: 88) | GGTTCTGTGCCCTGTCCAGC | I |
| Qs-Tg14-ASO-089 | (SEQ ID NO: 89) | GTGTGTTTCCTGCTCTCTCC | I |
| Qs-Tg14-ASO-090 | (SEQ ID NO: 90) | CCTGCCAGCCTTGTTCCTCT | I |
| Qs-Tg14-ASO-091 | (SEQ ID NO: 91) | GGGTGTCTCCTCCCAGGACC | I |
| Qs-Tg14-ASO-092 | (SEQ ID NO: 92) | CCCACCTCTTAGCCCTCTCC | I |
| Qs-Tg14-ASO-093 | (SEQ ID NO: 93) | AGCACTGGGCTGGCTCTCCT | I |
| Qs-Tg14-ASO-094 | (SEQ ID NO: 94) | GCTCTCCCTCCTACCAGGTC | I |
| Qs-Tg14-ASO-095 | (SEQ ID NO: 95) | TCCTCCCCAGAGGCCAGTTT | I |
| Qs-Tg14-ASO-096 | (SEQ ID NO: 96) | AGCCCTCAGTCCACCAGGCA | I |
| Qs-Tg14-ASO-097 | (SEQ ID NO: 97) | TCCCCCATCTCCCTAAGCCT | I |
| Qs-Tg14-ASO-098 | (SEQ ID NO: 98) | GTAGCTGCCACCCTTCCTGC | I |
| Qs-Tg14-ASO-099 | (SEQ ID NO: 99) | TTTGCCCCACCTCCCTCCCT | I |
| Qs-Tg14-ASO-100 | (SEQ ID NO: 100) | GCCTCTGCTTGCCTTCTCCC | I |
| Qs-Tg14-ASO-101 | (SEQ ID NO: 101) | GGCTGCTACTCCTCCCCACA | I |
| Qs-Tg14-ASO-102 | (SEQ ID NO: 102) | GCCTCCCTCTTAGCCACTGG | I |
| Qs-Tg14-ASO-103 | (SEQ ID NO: 103) | GCAGGCCCTCTTCCCTAAGT | I |
| Qs-Tg14-ASO-104 | (SEQ ID NO: 104) | GGTTGCTCTTTCCCTGTGGC | I |
| Qs-Tg14-ASO-105 | (SEQ ID NO: 105) | GCCCTGCAGCACCAGCACTC | I |
| Qs-Tg14-ASO-106 | (SEQ ID NO: 106) | CTCCCCTTCTCCCACAGCTT | I |
| Qs-Tg14-ASO-107 | (SEQ ID NO: 107) | TCCCCACCCTCTTTAGTCCC | I |
| Qs-Tg14-ASO-108 | (SEQ ID NO: 108) | GGCCCAGTCTCACTTTGGCT | I |
| Qs-Tg14-ASO-109 | (SEQ ID NO: 109) | AGTCCCCTGGGTCAGCTTCA | I |
| Qs-Tg14-ASO-110 | (SEQ ID NO: 110) | CCACACAAGCTGCCCCCTCC | I |
| Qs-Tg14-ASO-111 | (SEQ ID NO: 111) | CCCCACCTGGCTGATGCCCC | I |
| Qs-Tg14-ASO-112 | (SEQ ID NO: 112) | CCTCTGGTTTGCTCTCCTGC | I |
| Qs-Tg14-ASO-113 | (SEQ ID NO: 113) | AGTCAGGTCCCCTTTCCCCT | I |
| Qs-Tg14-ASO-114 | (SEQ ID NO: 114) | TCCTTCTGCCTGCCTGCCAG | I |
| Qs-Tg14-ASO-115* | (SEQ ID NO: 115) | TGGTGGTTTCTTGAGCTTGC | E |
| Qs-Tg14-ASO-116* | (SEQ ID NO: 116) | GGCCTTTGGTGGTTTCTTGA | E |
| Qs-Tg14-AS0-117* | (SEQ ID NO: 117) | TGTTTGGCTTGGGCTTGCTG | E |
| Qs-Tg14-AS0-118* | (SEQ ID NO: 118) | CCATCTATGCAGAAACCTCT | E |
| Qs-Tg14-ASO-119* | (SEQ ID NO: 119) | GGTGCCATTTTCAAGATAGC | E |
| Qs-Tg14-ASO-120* | (SEQ ID NO: 120) | CATACACTGGTATCCCTCTG | E |
| Qs-Tg14-ASO-121* | (SEQ ID NO: 121) | TCATACACTGGTATCCCTCT | E |
| Qs-Tg14-ASO-122* | (SEQ ID NO: 122) | TTCATACACTGGTATCCCTC | E |
| Qs-Tg14-ASO-123* | (SEQ ID NO: 123) | CTTTCATACACTGGTATCCC | E |
| Qs-Tg14-ASO-124* | (SEQ ID NO: 124) | GCTTTCATACACTGGTATCC | E |
| Qs-Tg14-ASO-125* | (SEQ ID NO: 125) | GGTTCCTTCCTGCTTTCATA | E |
| Qs-Tg14-ASO-126* | (SEQ ID NO: 126) | GCTTCTTAAGTTGCTCCAAC | E |
| Qs-Tg14-ASO-127* | (SEQ ID NO: 127) | ACCTTCTTCCTCTATGGCAT | E |
| Qs-Tg14-ASO-128* | (SEQ ID NO: 128) | CACCTTCTTCCTCTATGGCA | E |
| Qs-Tg14-ASO-129* | (SEQ ID NO: 129) | TGCACTCTTTGAGCTGAGTT | E |
| Qs-Tg14-ASO-130* | (SEQ ID NO: 130) | CTCCCTATTCTGTTGTCTGG | E |
| Qs-Tg14-ASO-131* | (SEQ ID NO: 131) | TTTCCACCTCAGTTGTAGCC | E |
| Qs-Tg14-ASO-132* | (SEQ ID NO: 132) | AGGAGGCTAATTGGTCCATG | E |
| Qs-Tg14-ASO-133* | (SEQ ID NO: 133) | TAGGAGGCTAATTGGTCCAT | E |
| Qs-Tg14-ASO-134* | (SEQ ID NO: 134) | CTGCTAGACTCAGTTCCATT | E |
| Qs-Tg14-ASO-135* | (SEQ ID NO: 135) | GAGTTCACAGTCCTGGTTGA | E |
| Qs-Tg14-ASO-136* | (SEQ ID NO: 136) | CAGCACCACCAAGTTGCCAA | E |
| Qs-Tg14-ASO-137* | (SEQ ID NO: 137) | TTCAGCACCACCAAGTTGCC | E |
| Qs-Tg14-ASO-138* | (SEQ ID NO: 138) | GGCCACACCCTTCTTGATAC | E |
| Qs-Tg14-ASO-139* | (SEQ ID NO: 139) | AGGCCACACCCTTCTTGATA | E |
| Qs-Tg14-ASO-140* | (SEQ ID NO: 140) | CAGGCCACACCCTTCTTGAT | E |
| Qs-Tg14-ASO-141* | (SEQ ID NO: 141) | CCAGGCCACACCCTTCTTGA | E |
| Qs-Tg14-ASO-142* | (SEQ ID NO: 142) | TGCACCTTTAGTTTGGTCCA | E |
| Qs-Tg14-ASO-143* | (SEQ ID NO: 143) | GCCATTGCCATTCTTCTGGA | E |
| Qs-Tg14-ASO-144* | (SEQ ID NO: 144) | GGAGCTGGTGTCATCTAGTT | E |
| Qs-Tg14-ASO-145* | (SEQ ID NO: 145) | CACAGGGACCTCTTCTACTT | E |
| Qs-Tg14-ASO-146* | (SEQ ID NO: 146) | TTCCACAGGGACCTCTTCTA | E |
| Qs-Tg14-ASO-147* | (SEQ ID NO: 16) | GTTGACCTGGCAGCACTTGA | E |
| Qs-Tg14-ASO-148* | (SEQ ID NO: 16) | GTTGACCTGGCAGCACTTGA | E |
| Qs-Tg14-ASO-149* | (SEQ ID NO: 41) | GGTGGTTTCTTGAGCTTGCT | E |
| Qs-Tg14-ASO-150* | (SEQ ID NO: 41) | GGTGGTTTCTTGAGCTTGCT | E |
| Qs-Tg14-ASO-151* | (SEQ ID NO: 44) | GCCATCTATGCAGAAACCTC | E |
| Qs-Tg14-ASO-152* | (SEQ ID NO: 44) | GCCATCTATGCAGAAACCTC | E |
| Qs-Tg14-ASO-153* | (SEQ ID NO: 45) | GCCTTTGGTGCCATTTTCAA | E |
| Qs-Tg14-ASO-154* | (SEQ ID NO: 45) | GCCTTTGGTGCCATTTTCAA | E |
| Qs-Tg14-ASO-155* | (SEQ ID NO: 117) | TGTTTGGCTTGGGCTTGCTG | E |
| Qs-Tg14-ASO-156* | (SEQ ID NO: 117) | TGTTTGGCTTGGGCTTGCTG | E |
| Qs-Tg14-ASO-157* | (SEQ ID NO: 124) | GCTTTCATACACTGGTATCC | E |
| Qs-Tg14-ASO-158* | (SEQ ID NO: 124) | GCTTTCATACACTGGTATCC | E |
| Qs-Tg14-ASO-159* | (SEQ ID NO: 126) | GCTTCTTAAGTTGCTCCAAC | E |
| Qs-Tg14-ASO-160* | (SEQ ID NO: 126) | GCTTCTTAAGTTGCTCCAAC | E |
| Qs-Tg14-ASO-161* | (SEQ ID NO: 129) | TGCACTCTTTGAGCTGAGTT | E |
| Qs-Tg14-ASO-162* | (SEQ ID NO: 129) | TGCACTCTTTGAGCTGAGTT | E |
| Qs-Tg14-ASO-163* | (SEQ ID NO: 133) | TAGGAGGCTAATTGGTCCAT | E |
| Qs-Tg14-ASO-164* | (SEQ ID NO: 133) | TAGGAGGCTAATTGGTCCAT | E |
| Qs-Tg14-ASO-165* | (SEQ ID NO: 135) | GAGTTCACAGTCCTGGTTGA | E |
| Qs-Tg14-ASO-166* | (SEQ ID NO: 135) | GAGTTCACAGTCCTGGTTGA | E |
| Qs-Tg14-ASO-167* | (SEQ ID NO: 138) | GGCCACACCCTTCTTGATAC | E |
| Qs-Tg14-ASO-168* | (SEQ ID NO: 138) | GGCCACACCCTTCTTGATAC | E |
| Qs-Tg14-ASO-169* | (SEQ ID NO: 139) | AGGCCACACCCTTCTTGATA | E |
| Qs-Tg14-ASO-170* | (SEQ ID NO: 139) | AGGCCACACCCTTCTTGATA | E |
| Qs-Tg14-ASO-171* | (SEQ ID NO: 142) | TGCACCTTTAGTTTGGTCCA | E |
| Qs-Tg14-ASO-172* | (SEQ ID NO: 142) | TGCACCTTTAGTTTGGTCCA | E |
| Qs-Tg14-ASO-173* | (SEQ ID NO: 143) | GCCATTGCCATTCTTCTGGA | E |
| Qs-Tg14-ASO-174* | (SEQ ID NO: 143) | GCCATTGCCATTCTTCTGGA | E |
| Qs-Tg14-ASO-175* | (SEQ ID NO: 144) | GGAGCTGGTGTCATCTAGTT | E |
| Qs-Tg14-ASO-176* | (SEQ ID NO: 144) | GGAGCTGGTGTCATCTAGTT | E |
| Qs-Tg16-ASO-003 | (SEQ ID NO: 147) | GGCCTTCCTTCTCATGCCAT | M |
| Qs-Tg16-ASO-004 | (SEQ ID NO: 148) | GCGAGCCTGGGATGCTGAGC | M |
| Qs-Tg16-ASO-009 | (SEQ ID NO: 149) | TACTTCTCCACGCTGCTGCC | M |
| Qs-Tg16-ASO-011 | (SEQ ID NO: 150) | GTGTCTGTCTCCACCATCAT | M |
| Qs-Tg16-ASO-013 | (SEQ ID NO: 151) | GTGCCTCCATTCTCCAGCTT | M |
| Qs-Tg16-ASO-014 | (SEQ ID NO: 152) | CCTTCCTCTGCCCGCTGCTG | M |
| Qs-Tg16-ASO-016 | (SEQ ID NO: 153) | GCAAGTCCTCCTCAGCCTGA | M |
| Qs-Tg16-ASO-017 | (SEQ ID NO: 154) | GCCTGAGCAAGTCCTCCTCA | M |
| Qs-Tg16-ASO-019 | (SEQ ID NO: 155) | TCGACCTGCTGTCCTGGTGA | M |
| Qs-Tg16-ASO-020 | (SEQ ID NO: 156) | TTCTCGACCTGCTGTCCTGG | M |

Other features and embodiments are within the scope of the disclosure. Embodiments of the disclosure include oligonucleotides, including locked nucleic acid (LNA) antisense oligonucleotides targeting SCN8A which are capable of inhibiting the expression of Nav1.6. The oligonucleotide of the invention may be used in the prevention or treatment of a condition such as epilepsy or pain. The invention further provides advantageous target site sequences on the human Nav1.6 pre-mRNA which may be targeted by oligonucleotide inhibitors of human Nav1.6 such as antisense oligonucleotides or RNAi agents, such as siRNAs or shRNAs.

The invention provides for an oligonucleotide of 10 to 30 nucleotides in length, which comprises a contiguous nucleotide sequence of 10 to 30 nucleotides in length with at least 90% complementarity, preferably 100% complementarity, to a human Nav1.6 RNA. An oligonucleotide 107 may be 100% identical to one of SEQ ID NO 1-156, or preferably at least 90% identical.

Embodiments include a pharmaceutically acceptable salt of the antisense oligonucleotide according to the invention, or the conjugate according to the invention.

The invention provides a pharmaceutical composition comprising the antisense oligonucleotide of the invention or the conjugate of the invention and a pharmaceutically acceptable diluent, solvent, carrier, salt and/or adjuvant.

The invention provides for the antisense oligonucleotide of the invention or the conjugate of the invention or the pharmaceutical salt or composition of the invention for use in medicine.

The invention provides for the antisense oligonucleotide of the invention or a pharmaceutical salt thereof. The invention provides for the use of the antisense oligonucleotide of the invention in the preparation of a medicament for the treatment, prevention or alleviation of a condition such as epilepsy or pain.

Oligonucleotides may be made in the laboratory by solid-phase chemical synthesis followed by purification and isolation. When referring to a sequence of the oligonucleotide, reference is made to the sequence or order of nucleobase moieties, or modifications thereof, of the covalently linked nucleotides or nucleosides. The oligonucleotide of the invention may be man-made, i.e., chemically synthesized, and is typically purified or isolated. The oligonucleotide of the invention may comprise one or more modified nucleosides or nucleotides, such as 2′ sugar modified nucleosides.

The modified nucleotides may be independently selected from the group consisting of a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, a 2′-hydroxl-modified nucleotide, a 2′-methoxyethyl modified nucleotide, a 2′-O-alkyl-modified nucleotide, a morpholino nucleotide, a phosphoramidate, a non-natural base comprising nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a glycol modified nucleotide, and a 2-O—(N-methylacetamide) modified nucleotide, and combinations thereof.

The nitrogenous bases of the ASO maybe naturally occurring nucleobases such as adenine, guanine, cytosine, thymidine, uracil, xanthine and hypoxanthine, as well as non-naturally occurring variants, such as substituted purine or substituted pyrimidine, such as nucleobases selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′-thio-thymine, inosine, diaminopurine, 6-aminopurine, 2-aminopurine, 2,6-diaminopurine and 2-chloro-6-aminopurine. In certain preferred embodiments, a composition of the invention includes an oligonucleotide in which some, many, most, or all cytosine bases are present in a methylated form, e.g., 5-methyl cytosine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

An oligonucleotide 107 of the disclosure is capable of down-regulating (inhibiting) the expression of a sodium channel (Nav1.6). In some embodiments the antisense oligonucleotide of the invention is capable of modulating the expression of the target by inhibiting or down-regulating it. Preferably, such modulation produces an inhibition of expression of at least 20% compared to the normal expression level of the target, more preferably at least 30%, at least 40%, at least 50%, at least 60%, at least 70% inhibition compared to the normal expression level of the target.

An antisense oligonucleotide of the disclosure may decrease the level of the target nucleic acid (e.g. via RNase H cleavage), or may decrease the functionality (or alter the functionality) of the target nucleic acid, e.g. via modulation of splicing of a pre-mRNA.

An oligonucleotide 107 of the disclosure may comprise one or more nucleosides which have a modified sugar moiety, i.e. a modification of the sugar moiety when compared to the ribose sugar moiety found in DNA and RNA. Numerous nucleosides with modification of the ribose sugar moiety have been made, primarily with the aim of improving certain properties of oligonucleotides, such as affinity and/or nuclease resistance. Such modifications include those where the ribose ring structure is modified, e.g. by replacement with a hexose ring (HNA), or a bicyclic ring, which typically have a bridge between the C2 and C4 carbons on the ribose ring (LNA), or an unlinked ribose ring which typically lacks a bond between the C2 and C3 carbons (e.g. UNA). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

The oligonucleotide may include one or more Locked Nucleic Acid (LNA) bases. An LNA may include a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be determined by measuring the melting temperature of the oligonucleotide/complement duplex. Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, and WO 2008/150729, all incorporated by reference.

Pharmaceutically acceptable salts of oligonucleotides of the disclosure include those salts that retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, a sulfonic acid, or salicylic acid. In addition, those salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins.

An oligonucleotide 107 may mediate or promote nuclease mediated degradation of sodium channel pre-mRNA or mRNA transcripts. Nuclease mediated degradation refers to an oligonucleotide capable of mediating degradation of a complementary nucleotide sequence when forming a duplex with such a sequence. In some embodiments, the oligonucleotide may function via nuclease mediated degradation of the target nucleic acid, where the oligonucleotides of the invention are capable of recruiting a nuclease, particularly an endonuclease, preferably endoribonuclease (RNase), such as RNase H. Examples of oligonucleotide designs which operate via nuclease mediated mechanisms are oligonucleotides which typically comprise a region of at least 5 or 6 consecutive DNA nucleosides and are flanked on one side or both sides by affinity enhancing nucleosides, for example gapmers. The RNase H activity of an antisense oligonucleotide 107 refers to its ability to recruit RNase H when in a duplex with a complementary RNA molecule.

The antisense oligonucleotide 107 of the invention, or contiguous nucleotide sequence thereof, may be a gapmer, also termed gapmer oligonucleotide or gapmer designs. The antisense gapmers are commonly used to inhibit a target nucleic acid via RNase H mediated degradation. A gapmer oligonucleotide comprises at least three distinct structural regions: a 5′-flank, a gap and a 3′-flank, F-G-F′ in the ‘5->3’ orientation. The “gap” region (G) comprises a stretch of contiguous DNA nucleotides which enable the oligonucleotide to recruit RNase H. The gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the oligonucleotide for the target nucleic acid (i.e. are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

A mixed wing gapmer may be an LNA gapmer wherein one or both of region F and F′ comprise a 2′ substituted nucleoside, such as a 2′ substituted nucleoside independently selected from the group consisting of 2′-O-alkyl-RNA units, 2′-O-methyl-RNA, 2′-amino-DNA units, 2′-fluoro-DNA units, 2′-alkoxy-RNA, 2′-MOE units, arabino nucleic acid (ANA) units, 2′-fluoro-ANA units, or combinations thereof. In some embodiments wherein at least one of region F and F′, or both region F and F′ comprise at least one LNA nucleoside, the remaining nucleosides of region F and F′ are independently selected from the group consisting of 2′-MOE and LNA. In some embodiments wherein at least one of region F and F′, or both region F and F′ comprise at least two LNA nucleosides, the remaining nucleosides of region F and F′ are independently selected from the group consisting of 2′-MOE and LNA. In some mixed wing embodiments, one or both of region F and F′ may further comprise one or more DNA nucleosides. Gapmer designs are discussed in WO 2008/049085 and WO 2012/109395, both incorporated by reference.

Conjugation of the oligonucleotide 107 to one or more non-nucleotide moieties may improve the pharmacology of the oligonucleotide, e.g. by affecting the activity, cellular distribution, cellular uptake or stability of the oligonucleotide. In some embodiments the conjugate moiety can modify or enhance the pharmacokinetic properties of the oligonucleotide by improving cellular distribution, bioavailability, metabolism, excretion, permeability, and/or cellular uptake of the oligonucleotide. In particular, the conjugate may target the oligonucleotide to a specific organ, tissue or cell type and thereby enhance the effectiveness of the oligonucleotide in that organ, tissue or cell type. The conjugate may also serve to reduce activity of the oligonucleotide in non-target cell types, tissues or organs, e.g. off target activity or activity in non-target cell types, tissues or organs.

In an embodiment, the non-nucleotide moiety (conjugate moiety) is selected from the group consisting of carbohydrates, cell surface receptor ligands, drug substances, hormones, lipophilic substances, polymers, proteins, peptides, toxins (e.g. bacterial toxins), vitamins, viral proteins (e.g. capsids) or combinations thereof.

Oligonucleotides 107 of the disclosure may be provided in pharmaceutical compositions that include any of the aforementioned oligonucleotides and/or oligonucleotide conjugates or salts thereof and a pharmaceutically acceptable diluent, carrier, salt and/or adjuvant. A pharmaceutically acceptable diluent includes artificial cerebrospinal fluid (ACSF) and pharmaceutically acceptable salts include, but are not limited to, sodium and potassium salts. In some embodiments the pharmaceutically acceptable diluent is sterile phosphate buffered saline or sterile sodium carbonate buffer. In some preferred embodiments, diluents for clinical application include Elliotts B solution and/or ACSF artificial cerebrospinal fluid.

In some embodiments the oligonucleotide of the invention is in the form of a solution in the pharmaceutically acceptable diluent, for example dissolved in phosphate buffered saline (PBS) or sodium carbonate buffer. The oligonucleotide may be pre-formulated in the solution or in some embodiments may be in the form of a dry powder (e.g. a lyophilized powder) which may be dissolved in the pharmaceutically acceptable diluent prior to administration. Suitably, for example the oligonucleotide may be dissolved in a concentration of 0.1-100 mg/mL, such as 1-10 mg/mL.

Compositions of the disclosure may be administered to a patient for the prevention or treatment of a condition such as epilepsy or pain such as chronic pain, neuropathic pain, inflammatory pain, spontaneous pain, or nociceptive pain. Preferred embodiments are used for the treatment of DEE13, Dravet syndrome, or epilepsy involving a pathogenic mechanism of excessive E/I balance ratio. The oligonucleotides of the invention, or the conjugates, salts or pharmaceutical compositions of the invention may be for use as a local analgesic.

The disclosure provides methods for treating or preventing a condition in a subject, such as a human, who is suffering from or is likely affected by the condition, the methods comprising administering a therapeutically or prophylactically effective amount of a composition of the disclosure, e.g., comprising an oligonucleotide as described herein to a subject who is suffering from or affected by a condition such as epilepsy or pain, such as cancer pain, osteoarthritis pain, chronic pain, neuropathic pain, inflammatory pain, spontaneous pain, or nociceptive pain, where the oligonucleotide is targeted to a sequence complementary to one of SEQ ID NOs: 1-114 according to any of the descriptions herein with any combination of the features described herein.

### Examples

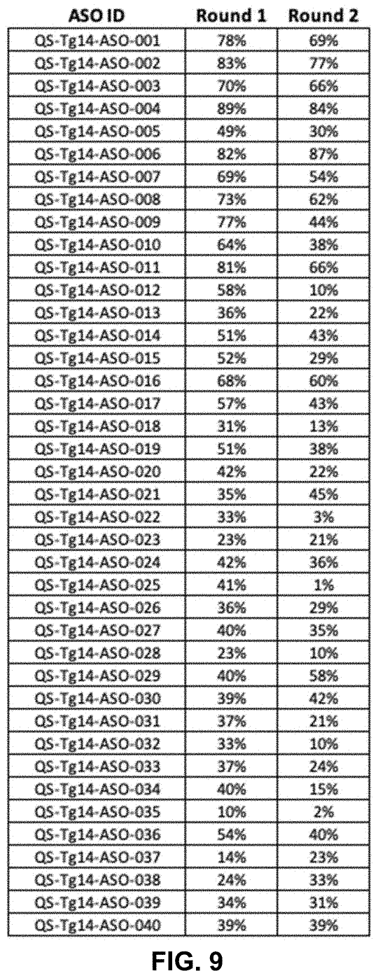

FIG. 9 through FIG. 11 give summary data of knockdown percent from single dose screening of SCN8A ASOs. SCN8A-targeting ASOs were screened in vitro by treating SK-N-AS neuroblastoma cells, plated at 20,000 cells per well of a 96-well plate, with 100 nM of ASO. Data from two rounds (replicates) are shown for each figure. ASOs were delivered by transfection using RNAiMax at 0.3 uL per well of a 96-well plate. The data shown displays a summary table of qPCR readouts of SCN8A knockdown (expressed as percent of SCN8A knockdown) for 146 ASOs screened in our primary screen. All samples were normalized to vehicle only (i.e. RNAiMax only) conditions.

FIG. 9 shows SCN8A exon-targeting ASOs targeting the full transcript.

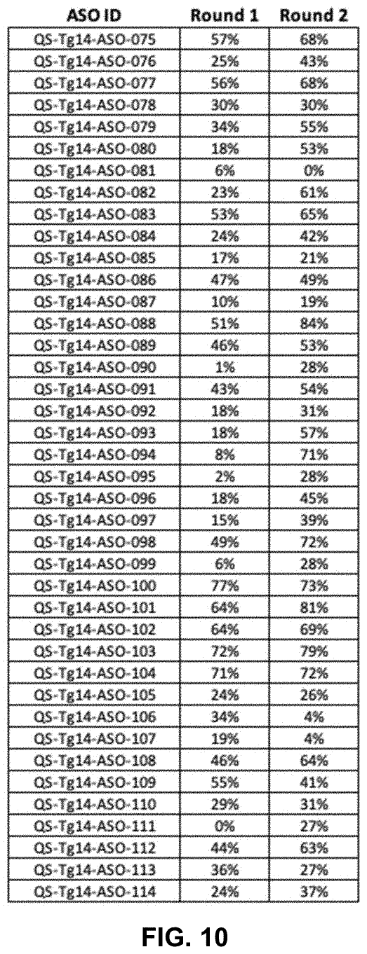

FIG. 10 shows SCN8A intron-targeting ASOs.

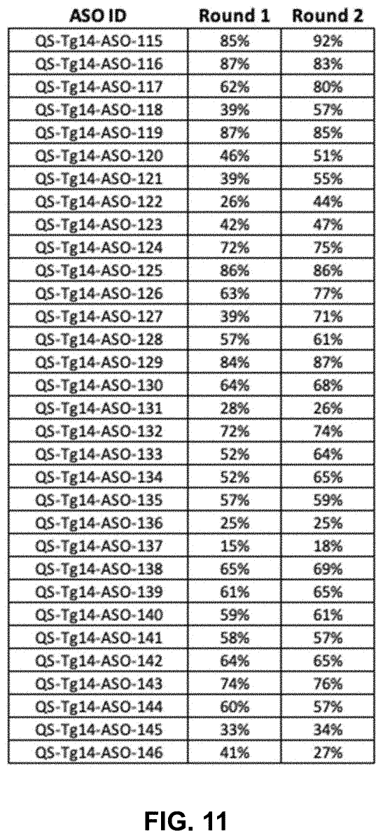

FIG. 11 gives results from optimized SCN8A exon-targeting ASOs focused on the first 3700 nucleotides of the transcript. All cells were transfected with ASOs at the time of plating and harvested for qPCR 48 hours after ASO transfection. Transcript levels for the housekeeping gene ACTIN were used to normalize levels for SCN8A. All data are shown across two independent rounds of plating, ASO treatment, and qPCRs.

Note: ASOs with the IDs ending in 115-146 were designed after screening of ASOs with the IDs ending in 001-074 in which we identified the first 3700 nucleotides of the SCN8A transcript (NM_014101.4) as a hotspot for modulation by ASOs. In other words, a larger number of ASOs targeting this region were successful in knocking down the SCN8A transcript by at least about 60%. The sequence for this portion of the transcript is available at GenBank under accession number NM_014101.4.

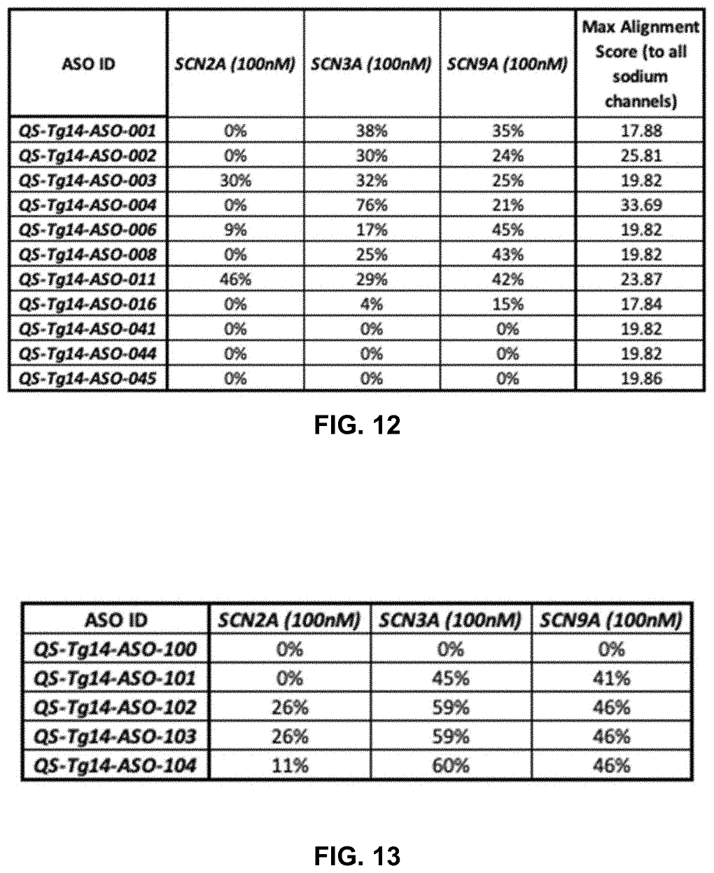

FIG. 12 through FIG. 14 give summary data of sodium channel counter-screen with SCN8A ASOs achieving at least 60% target knockdown in single dose screen

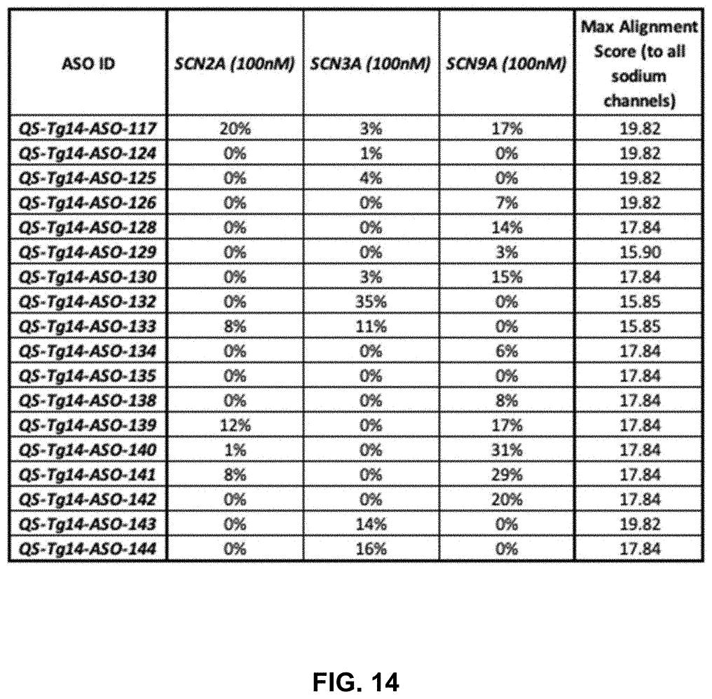

From the single dose screening experiments (shown in FIG. 9 through FIG. 11), ASOs were prioritized which achieved at least 60% SCN8A transcript knockdown in SK-N-AS cells. Due to known homology in transcript sequences across sodium channels, ASO candidates were further screened to quantify their ability to knock down other sodium channels (which is not a desired characteristic of a lead candidate). Experimental focus via qPCR was on SCN2A, SCN3A, and SCN9A which are known to be expressed in this cell type. For other sodium channels not expressed in SK-N-AS cells, a computational alignment score (righthand column, FIG. 12 and FIG. 14) was calculated which predicts on a scale of approximately 11 to 40 the potential for off-target effects of the ASOs (where 40 represents a perfect match). Here, qPCR knockdown and maximum predicted alignment scores across all sodium channels are shown for prioritized ASOs

FIG. 12 gives the data from SCN8A exon-targeting ASOs targeting the full transcript.

FIG. 13 gives the data from SCN8A intron-targeting ASOs.

FIG. 14 gives the data from optimized SCN8A exon-targeting ASOs focused on the first 3700 nucleotides of the transcript. Since predicted alignments to homologous sodium channels was considered in the design of ASOs 115-146, there are lower levels of off-target knockdown overall in this batch. Note that maximum predicted alignment scores are not shown for intron-targeting ASOs since they are based on alignments to exons.

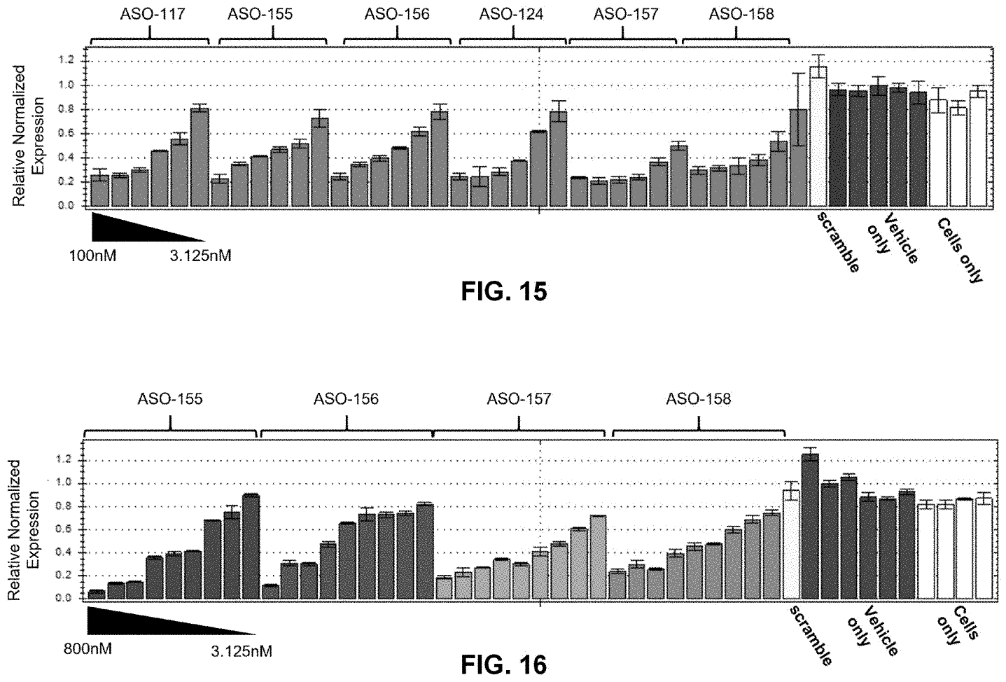

FIG. 15 and FIG. 16 give examples of dose-response screening of SCN8A lead ASO candidates

Candidate lead SCN8A-targeting ASOs were selected based on at least 60% SCN8A transcript knockdown in single-dose screening (FIG. 9-11) and less than about 20% transcript knockdown in homologous sodium channels (FIG. 1012-14). For each candidate lead sequence, new ASOs with identical sequences, were synthesized with 1 to 3 PO backbone modifications each in the 3′ and 5′, 2′-MOE RNA-like wings (total of 3-4 PO modifications per ASO). These candidate leads were then tested for dose-response modulation of SCN8A transcript expression. For these experiments, SK-N-AS neuroblastoma cells, plated at density of 20,000 cells per well of a 96-well plate, were treated at a range of concentrations: 800, 400, 200, 100, 50, 25, 12.5, 6.25, and 3.125 nM. ASOs were delivered by transfection using RNAiMax at 0.3 uL per well of a 96-well plate. All cells were transfected with ASOs at the time of plating and harvested for qPCR 48 hours after ASO transfection. ACTIN was used as the normalizing gene for SCN8A. Each data point represents two technical replicates and one biological replicate.

FIG. 15 shows five-point dose-response data for two lead SCN8A ASO parent candidates (117, 124) and their PO-modified daughter molecules (155, 156, 157, 158) in SK-N-AS neuroblastoma cells.

FIG. 16 shows nine-point dose-response data for the same PO-modified daughter molecules.

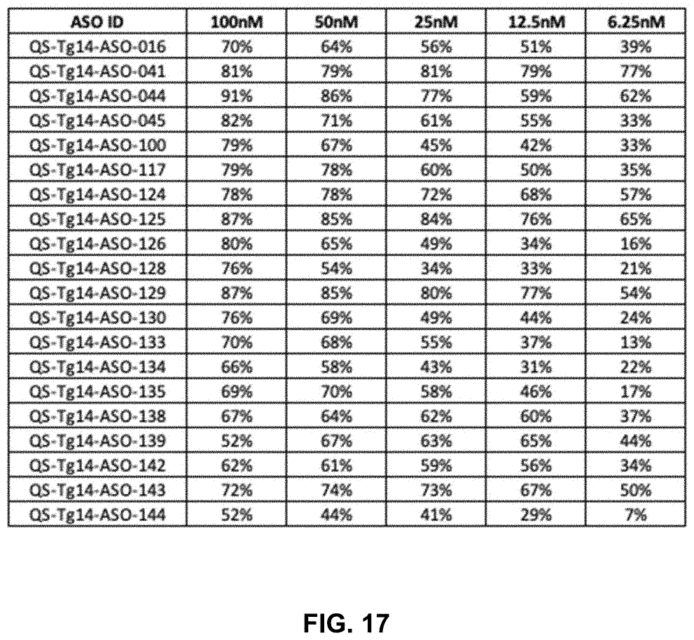

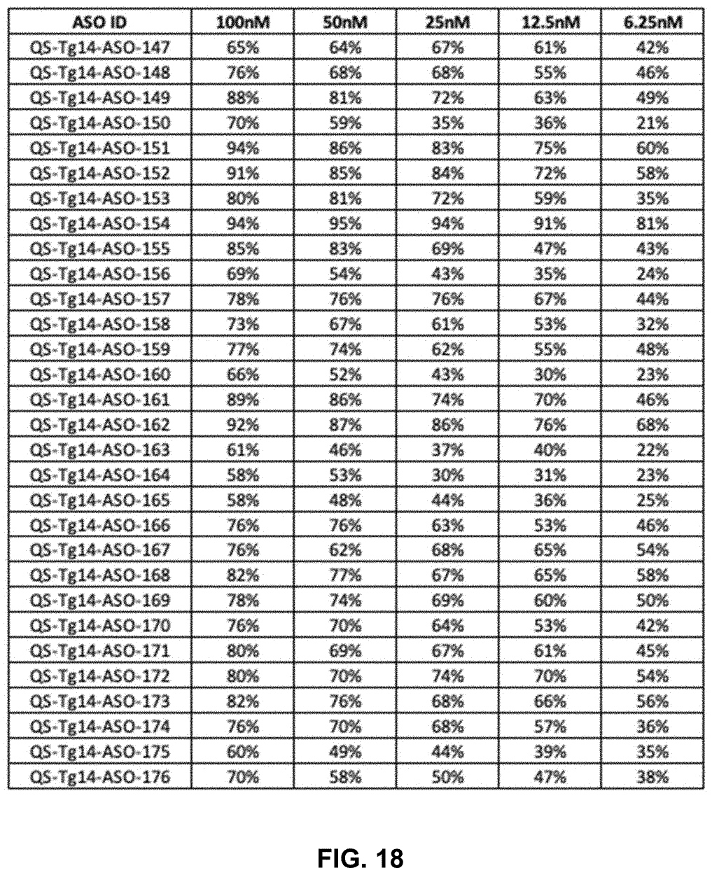

FIG. 17 and FIG. 18 give summary data of dose-response screening of SCN8A ASO lead candidates.

Candidate lead SCN8A-targeting ASOs were selected based on at least 60% transcript knockdown in the primary single-dose screening. For each candidate lead, new ASOs with identical sequences, were synthesized with 1 to 3 PO backbone modifications each in the 3′ and 5′, 2′-MOE RNA-like wings (total of 3-4 PO modifications per ASO). All candidate leads were then tested for dose-response modulation of SCN8A transcript expression. For these experiments, SK-N-AS neuroblastoma cells, plated 20 k per well of a 96-well plate, were plated onto a 96-well plate. ASOs were screened at 5 doses: 100, 50, 25, 12.5, 6.25 nM. ASOs were delivered by transfection using RNAiMax at 0.3 uL per well of a 96-well plate. All cells were transfected with ASOs at the time of plating and harvested for qPCR 48 hours after ASO transfection. Actin was used as the normalizing gene for SCN8A. All samples were further normalized to vehicle conditions within each experiment. Dose-response data for all lead candidates are displayed and broken down.

FIG. 17 shows the data for the lead all-PS backbone candidates targeting SCN8A exons.

FIG. 18 shows the data for PO-modified daughter leads for human clinical candidates.

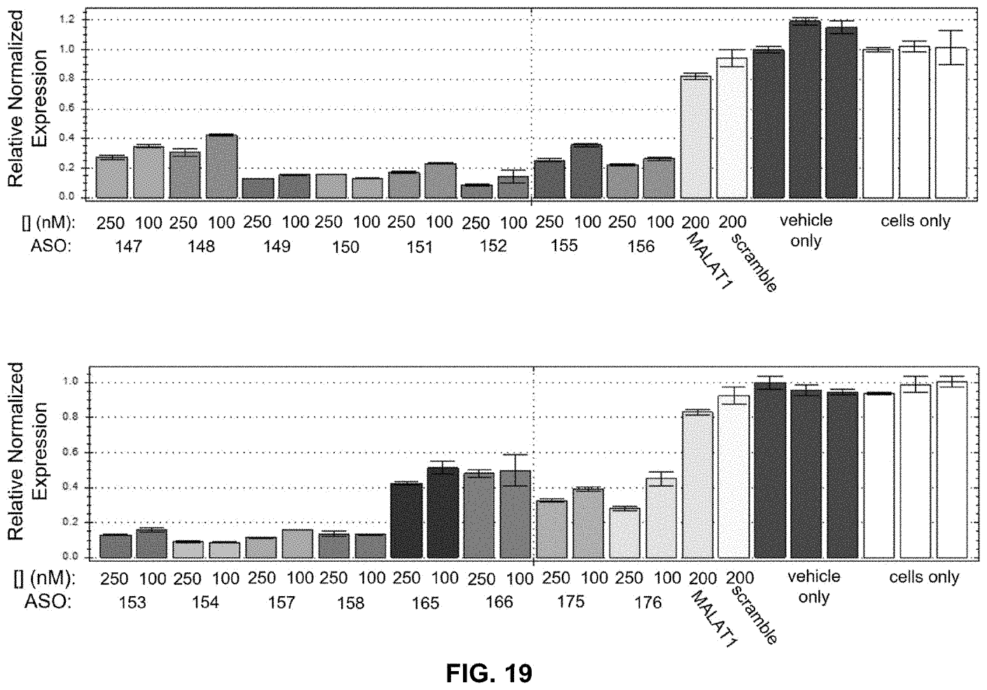

FIG. 19 shows knockdown of SCN8A transcript in human NGN2 stem cell-derived neurons using SCN8A lead candidates

SCN8A is critical in the excitability of neurons and, consequently this cell type is important for assessing the functional effects of target transcript knockdown. To show that our ASOs are effective in a relevant human cell type, we transfected human induced pluripotent stem cell-derived neurons (differentiated via NGN2 overexpression and dual SMAD inhibition) with our SCN8A ASOs. Neurons were plated at 70 k per well on a 96-well plate and treated with 250 and 100 nM SCN8A ASO. ASOs were transfected into neurons with Endoporter PEG transfection reagent (0.6 uL per well) on DIV20. Cells were harvested for qPCR 4 days after treatment on DIV24. Many ASOs show >80% knockdown of SCN8A transcript in human neurons. Beta tubulin was used as the normalizing gene for SCN8A. Each bar represents two technical replicates and one biological replicate.

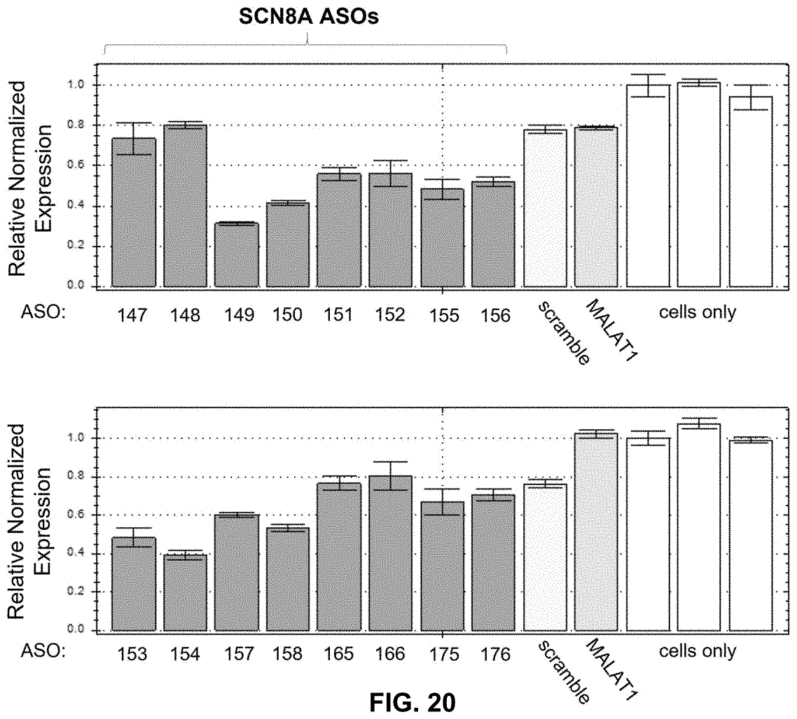

FIG. 20 shows knockdown of SCN8A transcript in human primary neurons using SCN8A lead candidates.

SCN8A is critical in the excitability of neurons and, consequently this cell type is important for assessing the functional effects of target transcript knockdown. To show that our ASOs are effective in a relevant human cell type, we transfected human primary neurons (derived from a 19-week old female fetus; acquired from ScienCell) with selected SCN8A ASOs. Neurons were plated at 30 k per well on a 96-well plate and treated with 1 uM SCN8A ASO. ASOs were delivered gymnotically on DIV1. Cells were harvested for qPCR 13 days after ASO treatment. Many ASOs show 50-60% knockdown of SCN8A transcript in human primary neurons with gymnotic delivery. Beta tubulin was used as the normalizing gene for SCN8A. Each bar represents two technical replicates and one biological replicate.

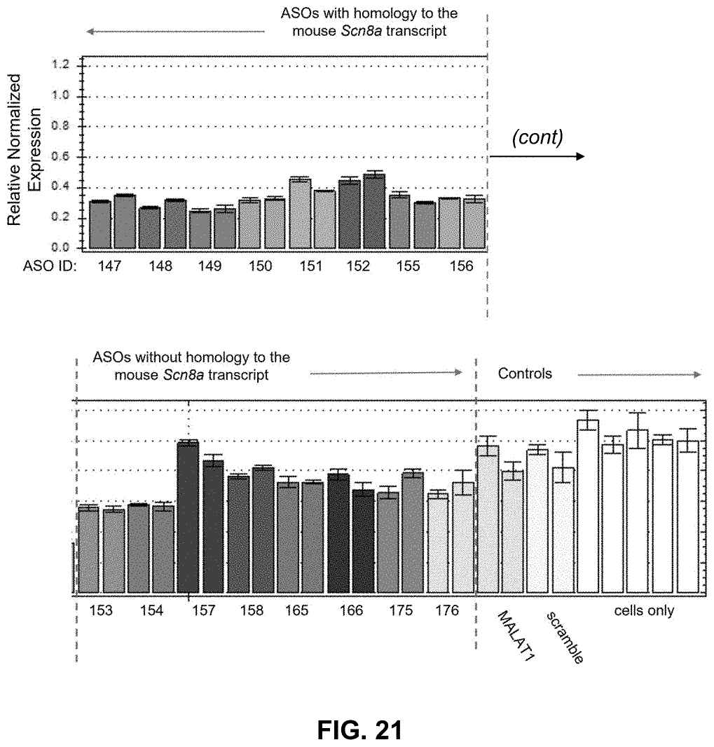

FIG. 21 shows knockdown of Scn8a transcript in mouse primary cortical neurons using SCN8A lead candidates

Two mouse models of SCN8A-caused encephalopathy are available (references below) carrying the mutations for R1872W and N1768D. These mouse models will be useful in showing proof-of-concept and efficacy in disease model systems in vivo. To show that selected ASOs are effective in a relevant mouse cell type, we treated mouse primary cortical neurons (Brainbits) with selected SCN8A ASOs. Neurons were plated at 50 k per well on a 96-well plate and treated with 500 nM SCN8A ASO. ASOs were delivered gymnotically on DIVS. Cells were harvested for qPCR 7 days after ASO treatment (on DIV12). SCN8A optimized lead candidates with backbone modifications were screened. ASOs with mouse homology show at least 60% knockdown. Beta tubulin was used as the normalizing gene for Scn8a. Each bar represents two technical replicates and one biological replicate.

An SCN8A-R1872W mouse model reference is described in Bunton-Stasyshyn, 2019, Prominent role of forebrain excitatory neurons in SCN8A encephalopathy, Brain 142(2): 362-375, incorporated by reference.

An SCN8A-N1768D mouse model reference is described in Wagnon, 2015, Convulsive seizures and SUDEP in a mouse model of SCN8A epileptic encephalopathy, Hum Mol Genet 24(2): 506-515, incorporated by reference.

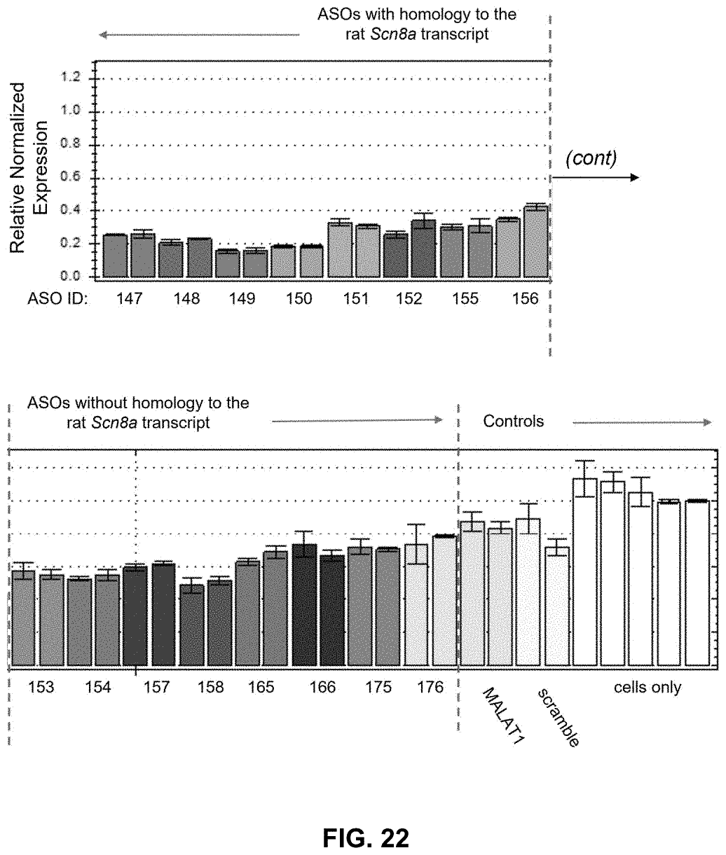

FIG. 22 shows knockdown of Scn8a transcript in rat primary hippocampal neurons using SCN8A lead candidates.

Lead ASOs are screened in vivo in rats to test for tolerability, toxicology, PK, and PD. To show that selected ASOs are effective in a relevant rat cell type, rat primary hippocampal neurons (Brainbits) were treated with selected SCN8A ASOs. Neurons were plated at 12 k per well on a 96-well plate and treated with 450 nM SCN8A ASO. ASOs were delivered gymnotically on DIVS. Cells were harvested for qPCR on DIV12, 7 days after ASO treatment. SCN8A optimized lead candidates with backbone modifications were screened. ASOs with rat homology show at least 60% knockdown. Beta tubulin was used as the normalizing gene for Scn8a. Each bar represents two technical replicates and one biological replicate.

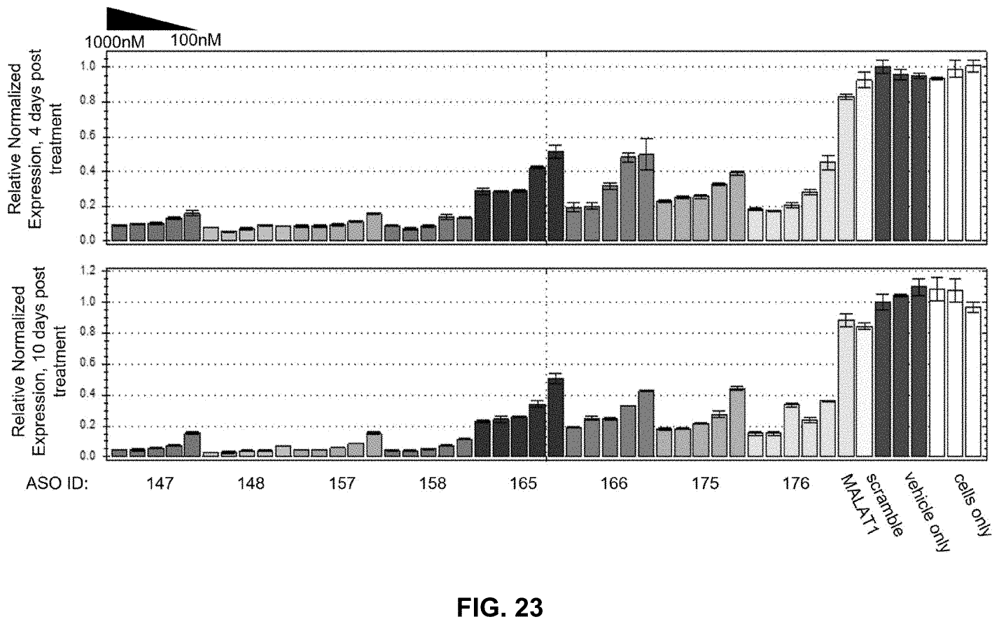

FIG. 23 shows evidence of a plateau in transcript knockdown in human NGN2 stem cell-derived neurons. It has been observed that Nav1.6 null mice die early. See Raman, 1997, Altered subthreshold sodium currents and disrupted firing patterns in Purkinje neurons of Scn8a Mutant Mice, Neuron, incorporated by reference. Literature suggests that hypomorphic mice expressing 10% of normal levels of Nav1.6 experience severe dystonia. See Kearney, 2002, Molecular and pathological effects of a modified gene on deficiency of the sodium channel Scn8a (Nav1.6), Hum Mol Genet, incorporated by reference. Evidence suggests that DEE13 mice treated with an ASO that knocks down Scn8a 50% experience decreased seizures and increased lifespan. See Lenk, 2020, Scn8a antisense oligonucleotide is protective in mouse models of SCN8A encephalopathy and Dravet syndrome, Ann Neurol, incorporated by reference. In view of that background, we hypothesize that the therapeutic window is within the range of 50-90% protein knockdown. An ASO with concentration response for SCN8A knockdown that achieves 50% knockdown and plateaus at a well-tolerated knockdown level of less than 90% may yield an optimal therapeutic profile.

We transfected human induced pluripotent stem cell-derived neurons (differentiated via NGN2 overexpression and dual SMAD inhibition) with our SCN8A ASOs. Neurons were plated at 70 k per well on a 96-well plate and treated in dose-response with 1000, 800, 500, 250 and 100 nM SCN8A ASO. ASOs were transfected into neurons with Endoporter PEG transfection reagent (0.6 uL per well) on DIV20. Cells were harvested for qPCR 4 and 10 days after treatment on DIV24 and DIV30. Beta tubulin was used as the normalizing gene for SCN8A. Each bar represents two technical replicates and one biological replicate.

ASOs designated 165, 166, 175, and 176 exhibit maximal knockdown of 70-80% beginning at 500 nM ASO treatment, whereas ASOs designated 153, 154, 157, and 158 exhibit >80% knockdown beginning at 100 nM.

ASO 165, aka 14-165, is SEQ ID NO: 135 in a gapmer with a 12 base DNA central segment flanked by 2′-MOE RNA wings, in which the 2d, 3rd, and 18th inter-base linkages are phosphodiester, with remaining inter-base linkages being phosphorothioate, and all cytosine bases having a 5-methyl modification. ASO 166, aka 14-166, is SEQ ID NO: 135 in a gapmer with a 12 base DNA central segment flanked by 2′-MOE RNA wings, in which 2d, 3rd, 4th, and 18th inter-base linkages are phosphodiester with remaining inter-base linkages being phosphorothioate, and all cytosine bases having a 5-methyl modification. ASO 175, aka 14-175, is SEQ ID NO: 144 in a gapmer with a 12 base DNA central segment flanked by 2′-MOE RNA wings, in which 2d, 3rd, and 18th inter-base linkages are phosphodiester with remaining inter-base linkages being phosphorothioate, and all cytosine bases having a 5-methyl modification. ASO 176, aka 14-176, is SEQ ID NO: 144 in a gapmer with a 12 base DNA central segment flanked by 2′-MOE RNA wings, in which 2d, 3rd, 4th, and 18th inter-base linkages are phosphodiester with remaining inter-base linkages being phosphorothioate, and all cytosine bases having a 5-methyl modification. The graph shows that these four compositions exhibit a maximal knockdown of about 70-80% for the concentration range tested (?).

SCN8A lead candidate ASOs for in vivo work are shown in in a vendor order format below (e.g., as one could order from a vendor such as Integrated DNA Technologies) and include ASO-147 through 158, 165, 166, 175, and 176. Unless otherwise shown, all ASOs are gapmers with a 12 base center and 3 to 5 base 2′-MOE RNA wings. All ASOs have mostly PS backbones except where indicated in the vendor order format. Cytosines are methylated.

Lead ASOs were selected based on single-dose and dose-response efficacy, sodium channel counter-screen data, sequence motif liabilities, and off-target alignment analyses. ASOs with the greatest in vitro efficacy to SCN8A, no knockdown in other sodium channels, lowest off-target alignments, and limited sequence motif concerns were prioritized as leads.

In Table 1, ASOs falling within the first 3700 nucleotides are indicated with an asterisk (*). Of those, ASOs with IDs ASO 14-001 through 14-146 and 16-003, 16-004, 16-009, 16-011, 16-013, 16-014, 16-016, 16-017, 16-019, and 16-020 were synthesized with the following chemistry: 4 (2′-MOE)×12 (DNA)×4 (2′-MOE); All C bases have 5-methyl modification; and all PS backbone.

The listing below shows certain SCN8A ASO candidates that have been synthesized. ASOs 14-147 through 176 were synthesized with modified linkages as follows: 2MOEr=2′-O-methoxyethyl RNA; i2MOEr=internal 2′-O-methoxyethyl RNA; iMe-dC/2MOErC/i2MOErC=5-methyl modification; and PS linkage (*) versus PO (//) linkage as shown in FASTA list.

As shown in the FASTA listing, certain embodiments use a gapmer ASO with a sequence given by at least one of SEQ ID NO: 16; SEQ ID NO: 41; SEQ ID NO: 44; SEQ ID NO: 45; SEQ ID NO: 117; SEQ ID NO: 124; SEQ ID NO: 126; SEQ ID NO: 129; SEQ ID NO: 133; SEQ ID NO: 135; SEQ ID NO: 138; SEQ ID NO: 139; SEQ ID NO: 142; SEQ ID NO: 143; or SEQ ID NO: 144, in which the gapmer has a 12 base DNA central segment flanked by 2′-MOE RNA wings, in which each wing has one or two phosphodiester linkages with remaining inter-base linkages being phosphorothioate, with all cytosine bases having a 5-methyl modification.

Certain most preferred embodiments (for a knockdown plateau achieving expression between about 50 and 90% normalized to untreated) use 14-165, 14-166, 14-175, or 14-176.

Here, 14-165 is SEQ ID NO: 135 in a gapmer with a 12 base DNA central segment flanked by 2′-MOE RNA wings, in which the 2d, 3rd, and 18th inter-base linkages are phosphodiester, with remaining inter-base linkages being phosphorothioate, and all cytosine bases having a 5-methyl modification. 14-166 is SEQ ID NO: 135 in a gapmer with a 12 base DNA central segment flanked by 2′-MOE RNA wings, in which 2d, 3rd, 4th, and 18th inter-base linkages are phosphodiester with remaining inter-base linkages being phosphorothioate, and all cytosine bases having a 5-methyl modification. 14-175 is SEQ ID NO: 144 in a gapmer with a 12 base DNA central segment flanked by 2′-MOE RNA wings, in which 2d, 3rd, and 18th inter-base linkages are phosphodiester with remaining inter-base linkages being phosphorothioate, and all cytosine bases having a 5-methyl modification. 14-176 is SEQ ID NO: 144 in a gapmer with a 12 base DNA central segment flanked by 2′-MOE RNA wings, in which 2d, 3rd, 4th, and 18th inter-base linkages are phosphodiester with remaining inter-base linkages being phosphorothioate, and all cytosine bases having a 5-methyl modification. The FASTA listing is a format that could be presented to a vendor such as Integrated DNA Technologies for ordering such ASOs.

Listing in vendor format:
>14-147*
(SEQ ID NO: 16)
/52MOErG/*/i2MOErT//i2MOErT//i2MOErG/*A*/iMe-dC/*/iMe-dC/*T*G* G*/
iMe-dC/*A* G*/iMe-dC/*A* /iMe-dC/*/i2MOErT/*/i2MOErT//i2MOErG/*/32MOErA/
>14-148*
(SEQ ID NO: 16)
/52MOErG/*/i2MOErT//i2MOErT//i2MOErG//i2MOErA/*/iMe-dC/* /iMe-dC/*T*G* G*/
iMe-dC/*A* G*/iMe-dC/*A* /iMe-dC/*T*/i2MOErT//i2MOErG/*/32MOErA/
>14-149*
(SEQ ID NO: 41)
/52MOErG/*/i2MOErG//i2MOErT//i2MOErG/*G*T*T*T*/iMe-dC/*T*T*G*A*G*/
iMe-dC/*T*/i2MOErT/*/i2MOErG//i2MOErC/*/32MOErT/
>14-150*
(SEQ ID NO: 41)
/52MOErG/*/i2MOErG//i2MOErT//i2MOErG//i2MOErG/*T*T*T*/
iMe-dC/*T*T*G*A*G*/iMe-dC/*T*T*/i2MOErG//i2MOErC/*/32MOErT/
>14-151*
(SEQ ID NO: 44)
/52MOErG/*/i2MOErC//i2MOErC//i2MOErA/*T*/iMe-dC/*T*A*T*G*/
iMe-dC/*A*G*A*A*A*/i2MOErC/*/i2MOErC//i2MOErT/*/32MOErC/
>14-152*
(SEQ ID NO: 44)
/52MOErG/*/i2MOErC//i2MOErC//i2MOErA//i2MOErT/*/iMe-dC/*T*A*T*G*/
iMe-dC/*A*G*A*A*A*/iMe-dC/*/i2MOErC//i2MOErT/*/32MOErC/
>14-153*
(SEQ ID NO: 45)
/52MOErG/*/i2MOErC//i2MOErC//i2MOErT/*T*T*G*G*T*G*/iMe-dC/*/
iMe-dC/*A*T*T*T*/i2MOErT/*/i2MOErC//i2MOErA/*/32MOErA/
>14-154*
(SEQ ID NO: 45)
/52MOErG/*/i2MOErC//i2MOErC//i2MOErT//i2MOErT/*T*G*G*T*G*/iMe-dC/*/
iMe-dC/*A*T*T*T*T*/i2MOErC//i2MOErA/*/32MOErA/
>14-155*
(SEQ ID NO: 117)
/52MOErT/*/i2MOErG//i2MOErT//i2MOErT/*T*G*G*/iMe-dC/*T*T*G*G*G*/
iMe-dC/*T*T*/i2MOErG/*/i2MOErC//i2MOErT/*/32MOErG/
>14-156*
(SEQ ID NO: 117)
/52MOErT/*/i2MOErG//i2MOErT//i2MOErT//i2MOErT/*G*G*/iMe-dC/*T*T*G*G*G*/
iMe-dC/*T*T*G*/i2MOErC//i2MOErT/*/32MOErG/
>14-157*
(SEQ ID NO: 124)
/52MOErG/*/i2MOErC//i2MOErT//i2MOErT/*T*/iMe-dC/*A*T*A*/iMe-dC/*A*/
iMe-dC/*T*G*G*T*/i2MOErA/*/i2MOErT//i2MOErC/*/32MOErC/
>14-158*
(SEQ ID NO: 124)
/52MOErG/*/i2MOErC//i2MOErT//i2MOErT//i2MOErT/*/iMe-dC/*A*T*A*/
iMe-dC/*A*/iMe-dC/*T*G*G*T*A*/i2MOErT//i2MOErC/*/32MOErC/
>14-159*
(SEQ ID NO: 126)
/52MOErG/*/i2MOErC//i2MOErT//i2MOErT/*/iMe-dC/*T*T*A*A*G*T*T*G*/iMe-
dC/*T*/iMe-dC/*/i2MOErC/*/i2MOErA//i2MOErA/*/32MOErC/
>14-160*
(SEQ ID NO: 126)
/52MOErG/*/i2MOErC//i2MOErT//i2MOErT//i2MOErC/*T*T*A*A*G*T*T*G*/
iMe-dC/*T*/iMe-dC/*/iME-dC/*/i2MOErA//i2MOErA/*/32MOErC/
>14-161*
(SEQ ID NO: 129)
/52MOErT/*/i2MOErG//i2MOErC//i2MOErA/*/iMe-dC/*T*/iMe-dC/
*_{T}*_{T}*_{T}*G*_{A}*G*/i_{Me}-d_{C}/*_{T}*G*/i2MOErA/*/i2MOErG//i2MOErT/*/32MOErT/
>14-162*
(SEQ ID NO: 129)
/52MOErT/*/i2MOErG//i2MOErC//i2MOErA//i2MOErC/*T*/iMe-dC/*T*T*T*G*A*G*/iMe-
dC/*T*G*A*/i2MOErG//i2MOErT/*/32MOErT/
>14-163*
(SEQ ID NO: 133)
/52MOErT/*/i2MOErA//i2MOErG//i2MOErG/*A*G*G*/iMe-
dC/*T*A*A*T*T*G*G*T*/i2MOErC/*/i2MOErC//i2MOErA/*/32MOErT/
>14-164*
(SEQ ID NO: 133)
/52MOErT/*/i2MOErA//i2MOErG//i2MOErG//i2MOErA/*G*G*/
iMe-dC/*T*A*A*T*T*G*G*T*/iMe-dC/*/i2MOErC//i2MOErA/*/32MOErT/
>14-165*
(SEQ ID NO: 135)
/52MOErG/*/i2MOErA//i2MOErG//i2MOErT/*T*/iMe-dC/*A*/iMe-dC/*A*G*T*/
iMe-dC/*/iMe-dC/*T*G*G*/i2MOErT/*/i2MOErT//i2MOErG/*/32MOErA/
>14-166*
(SEQ ID NO: 135)
/52MOErG/*/i2MOErA//i2MOErG//i2MOErT//i2MOErT/*/iMe-dC/*A*/
iMe-dC/*A*G*T*/iMe-dC/*/iMe-dC/*T*G*G*T*/i2MOErT//i2MOErG/*/32MOErA/
>14-167*
(SEQ ID NO: 138)
/52MOErG/*/i2MOErG//i2MOErC//i2MOErC/*A*/iMe-dC/*A*/iMe-dC/*/
iMe-dC/*/iMe-dC/*T*T*/iMe-dC/*T*T*G*/i2MOErA/*/i2MOErT//i2MOErA/*/32MOErC/
>14-168*
(SEQ ID NO: 138)
/52MOErG/*/i2MOErG//i2MOErC//i2MOErC//i2MOErA/*/iMe-dC/*A*/iMe-dC/*/iMe-
dC/*/iMe-dC/*T*T*/iMe-dC/*T*T*G*A*/i2MOErT//i2MOErA/*/32MOErC/
>14-169*
(SEQ ID NO: 139)
/52MOErA/*/i2MOErG//i2MOErG//i2MOErC/*/iMe-dC/*A*/iMe-dC/*A*/iMe-dC/*/
iMe-dC/*/iMe-dC/*T*T*/iMe-dC/*T*T*/i2MOErG/*/i2MOErA//i2MOErT/*/32MOErA/
>14-170*
(SEQ ID NO: 139)
/52MOErA/*/i2MOErG//i2MOErG//i2MOErC//i2MOErC/*A*/iMe-dC/*A*/
iMe-dC/*/iMe-dC/*/iMe-dC/*T*T*/iMe-dC/*T*T*G*/i2MOErA//i2MOErT/*/32MOErA/
>14-171*
(SEQ ID NO: 142)
/52MOErT/*/i2MOErG//i2MOErC//i2MOErA/*/iMe-dC/*/iMe-
_{dc/}*_{T}*_{T}*_{T}*_{A}*G*_{T}*_{T}*_{T}*G*G*/i2MOErT/*/i2MOErC//i2MOErC/*/32MOErA/
>14-172*
(SEQ ID NO: 142)
/52MOErT/*/i2MOErG//i2MOErC//i2MOErA//i2MOErC/*/
iMe-dC/*T*T*T*A*G*T*T*T*G*G*T*/i2MOErC//i2MOErC/*/32MOErA/
>14-173*
(SEQ ID NO: 143)
/52MOErG/*/i2MOErC//i2MOErC//i2MOErA/*T*T*G*/
iMe-dC/*/iMe-dC/*A*T*T*/iMe-dC/*T*T*/iMe-
dC/*/i2MOErT/*/i2MOErG//i2MOErG/*/32MOErA/
>14-174*
(SEQ ID NO: 143)
/52MOErG/*/i2MOErC//i2MOErC//i2MOErA//i2MOErT/*T*G*/iMe-dC/*/iMe-
dC/*A*T*T*/iMe-dC/*T*T*/iMe-dC/*T*/i2MOErG//i2MOErG/*/32MOErA/
>14-175*
(SEQ ID NO: 144)
/52MOErG/*/i2MOErG//i2MOErA//i2MOErG/*/
iMe-dC/*T*G*G*T*G*T*/iMe-dC/*A*T*/iMe-dC/*T*/
i2MOErA/*/i2MOErG//i2MOErT/*/32MOErT/
>14-176*
(SEQ ID NO: 144)
/52MOErG/*/i2MOErG//i2MOErA//i2MOErG//i2MOErC/*T*G*G*T*G*T*/
iMe-dC/*A*T*/iMe-dC/*T*A*/i2MOErG//i2MOErT/*/32MOErT/

What is claimed is:

1. A composition comprising:
an oligonucleotide that hybridizes to an RNA encoding a sodium channel protein along a segment of the RNA that is at least about 75% complementary to one of SEQ ID NOs: 1-156 to thereby prevent translation of the RNA into the sodium channel protein.

2. The composition of claim 1, wherein the oligonucleotide hybridizes to, and knocks down expression of, Nav1.6 pre-mRNA or mRNA.

3. The composition of claim 1, wherein a sequence of bases in the oligonucleotide has at least about 80% identity to one of SEQ ID NOs: 1-156.

4. The composition of claim 1, wherein a sequence of bases in the oligonucleotide is at least about 95% identical to one of SEQ ID NOs: 1-156, wherein the oligonucleotide can hybridize to, and induce RNase cleavage of, Nav1.6 pre-mRNA or mRNA.

5. The composition of claim 1, wherein the composition comprises a plurality of therapeutic oligonucleotides each having a base sequence at least about 80% identical to one of SEQ ID NOs: 1-156, wherein each of the therapeutic oligonucleotides has a gapmer structure comprising a central DNA segment flanked by modified RNA wings, wherein the plurality of therapeutic oligonucleotides are provided in a solution or carrier formulated for intrathecal injection.

6. The composition of claim 1, wherein the oligonucleotide comprises two wings flanking a central region of at least about 9 DNA bases.

7. The composition of claim 1, wherein at least one end of the oligonucleotide comprises modified RNA bases.

8. The composition of claim 7 wherein each modified RNA base is selected from the group consisting of 2′-O-methoxyethyl RNA and 2′-O-methyl RNA.

9. The composition of claim 1, wherein the oligonucleotide comprises at least about 15 bases.

10. The composition of claim 1, wherein the oligonucleotide comprises between about 15 and about 25 bases.

11. The composition of claim 1, wherein the oligonucleotide has a backbone comprising a plurality of phosphorothioate bonds.

12. The composition of claim 1, wherein the oligonucleotide has a base sequence that has been screened and determined to not meet a threshold match for any non-target transcripts in humans.

13. The composition of claim 1, wherein the composition comprises a plurality of copies of a therapeutic oligonucleotide having a base sequence at least about 95% identical to one of SEQ ID NOs: 1-156, wherein the therapeutic oligonucleotide has a gapmer structure comprising a central twelve-base DNA segment flanked by two wings of four 2′-O-methoxyethyl RNA bases and a backbone of phosphorothioate linkages.

14. The composition of claim 1, wherein when the composition is delivered to cells in vitro, the cells exhibit a dose-dependent knockdown of Nav1.6.

15. The composition of claim 1, wherein the oligonucleotide has a base sequence with at least about a 90% match to one of SEQ ID NO: 1-156, with bases linked only by phosphorothioate linkages, the oligonucleotide further comprising a central 12 DNA bases flanked by a 5′ wing and a 3′ wing, the 5′ wing and the 3′ wing each comprising four consecutive 2′ modified RNA bases.

16. The composition of claim 15, wherein the oligonucleotide has a base sequence with at least about a 90% match to one of SEQ ID NO: 16, 41, 44, 45, 100, 117, 124, 125, 126, 128, 129, 130, 133, 134, 135, 138, 139, 142, 143, and 144.

17. The composition of claim 1, wherein the oligonucleotide has a base sequence matching one of SEQ ID NO: 1-156, with a majority of inter-base linkages comprising phosphorothioate linkages, the oligonucleotide further comprising a central 12 DNA bases flanked by a 5′ wing and a 3′ wing, the 5′ wing and the 3′ wing each comprising four consecutive 2′-MOE RNA bases.

18. The composition of claim 17, wherein the oligonucleotide has a base sequence matching one of SEQ ID NO: 16, 41, 44, 45, 100, 117, 124, 125, 126, 128, 129, 130, 133, 134, 135, 138, 139, 142, 143, and 144.

19. The composition of claim 1, wherein the oligonucleotide hybridizes to a location within the first 3700 bases of an SCN8A transcript.

20. The composition of claim 1, wherein the oligonucleotide has a sequence of one selected from the group consisting of SEQ ID NO: 16; SEQ ID NO: 41; SEQ ID NO: 44; SEQ ID NO: 45; SEQ ID NO: 117; SEQ ID NO: 124; SEQ ID NO: 126; SEQ ID NO: 129; SEQ ID NO: 133; SEQ ID NO: 135; SEQ ID NO: 138; SEQ ID NO: 139; SEQ ID NO: 142; SEQ ID NO: 143; or SEQ ID NO: 144.

21. The composition of claim 20, further wherein the oligonucleotide is a gapmer with a 12 base DNA central segment flanked by two 2′-MOE RNA wings, in which each wing has one, two, or three phosphodiester linkages with remaining inter-base linkages being phosphorothioate, and wherein all cytosine bases have a 5-methyl modification.

22. The composition of claim 1, wherein the oligonucleotide knocks down expression of an SCN8A transcript to an amount about 50 to 90% compared to an un-treated control.

23. The composition of claim 1, further wherein the oligonucleotide is one selected from the group consisting of:
SEQ ID NO: 135 in a gapmer with a 12 base DNA central segment flanked by 2′-MOE RNA wings, in which the 2d, 3rd, and 18th inter-base linkages are phosphodiester with remaining inter-base linkages being phosphorothioate, and all cytosine bases having a 5-methyl modification;
SEQ ID NO: 135 in a gapmer with a 12 base DNA central segment flanked by 2′-MOE RNA wings, in which 2d, 3rd, 4th, and 18th inter-base linkages are phosphodiester with remaining inter-base linkages being phosphorothioate, and all cytosine bases having a 5-methyl modification;
SEQ ID NO: 144 in a gapmer with a 12 base DNA central segment flanked by 2′-MOE RNA wings, in which 2d, 3rd, and 18th inter-base linkages are phosphodiester with remaining inter-base linkages being phosphorothioate, and all cytosine bases having a 5-methyl modification; and
SEQ ID NO: 144 in a gapmer with a 12 base DNA central segment flanked by 2′-MOE RNA wings, in which 2d, 3rd, 4th, and 18th inter-base linkages are phosphodiester with remaining inter-base linkages being phosphorothioate, and all cytosine bases having a 5-methyl modification.

24. The composition of claim 23, wherein the oligonucleotide knocks down expression of an SCN8A transcript to an amount about 50 to 70% when delivered to cells at about 1000 nM concentration compared to a control.

25. A method comprising: administering to a subject with epilepsy a composition of claim 1 to thereby knockdown expression of a SCN8A gene.

26. The method of claim 25, wherein the epilepsy comprises Dravet syndrome, DEE13, or an epilepsy involving a pathogenic mechanism of excessive E/I balance.


SEQUENCE LISTING (156 sequences)

SEQ ID NO: 1 - DNA, 20 bp, Artificial Sequence
  antisense oligonucleotide
gaagagagttttccctctgt

SEQ ID NO: 2 - DNA, 20 bp, Artificial
  antisense oligonucleotide
ggtatccctctgggcattgc

SEQ ID NO: 3 - DNA, 20 bp, Artificial
  antisense oligonucleotide
gggttccttcctgctttcat

SEQ ID NO: 4 - DNA, 20 bp, Artificial
  antisense oligonucleotide
agcctctttttgttctgcct

SEQ ID NO: 5 - DNA, 20 bp, Artificial
  antisense oligonucleotide
gcagcctgtgcctcttcctg

SEQ ID NO: 6 - DNA, 20 bp, Artificial
  antisense oligonucleotide
aggccttccttctcatgcca

SEQ ID NO: 7 - DNA, 20 bp, Artificial
  antisense oligonucleotide
gcactttctctgagactctt

SEQ ID NO: 8 - DNA, 20 bp, Artificial
  antisense oligonucleotide
ggcactttctctgagactct

SEQ ID NO: 9 - DNA, 20 bp, Artificial
  antisense oligonucleotide
ccagtgtcaggttgcccagg

SEQ ID NO: 10 - DNA, 20 bp, Artificial
  antisense oligonucleotide
gggagttcacagtcctggtt

SEQ ID NO: 11 - DNA, 20 bp, Artificial
  antisense oligonucleotide
ggcctgccacttccatgcag

SEQ ID NO: 12 - DNA, 20 bp, Artificial
  antisense oligonucleotide
ggtccaggccacacccttct

SEQ ID NO: 13 - DNA, 20 bp, Artificial
  antisense oligonucleotide
tcctctgtgttgaggttctc

SEQ ID NO: 14 - DNA, 20 bp, Artificial
  antisense oligonucleotide
ggctgttccacagggacctc

SEQ ID NO: 15 - DNA, 20 bp, Artificial
  antisense oligonucleotide
gacctggcagcacttgaacc

SEQ ID NO: 16 - DNA, 20 bp, Artificial
  antisense oligonucleotide
gttgacctggcagcacttga

SEQ ID NO: 17 - DNA, 20 bp, Artificial
  antisense oligonucleotide
tgttgacctggcagcacttg

SEQ ID NO: 18 - DNA, 20 bp, Artificial
  antisense oligonucleotide
gacttgcctagcccttcctc

SEQ ID NO: 19 - DNA, 20 bp, Artificial
  antisense oligonucleotide
gatgagacacaccagcagca

SEQ ID NO: 20 - DNA, 20 bp, Artificial
  antisense oligonucleotide
tctctgtattgttcccctcc

SEQ ID NO: 21 - DNA, 20 bp, Artificial
  antisense oligonucleotide
gccaggtatcctgccccaac

SEQ ID NO: 22 - DNA, 20 bp, Artificial
  antisense oligonucleotide
atgaagatgtcctgacctcc

SEQ ID NO: 23 - DNA, 20 bp, Artificial
  antisense oligonucleotide
gtggcttctttgagcccagc

SEQ ID NO: 24 - DNA, 20 bp, Artificial
  antisense oligonucleotide
atgttctccatctgcttgct

SEQ ID NO: 25 - DNA, 20 bp, Artificial
  antisense oligonucleotide
gcaggccatcccaaccagct

SEQ ID NO: 26 - DNA, 20 bp, Artificial
  antisense oligonucleotide
gggtctgcactttcctctgt

SEQ ID NO: 27 - DNA, 20 bp, Artificial
  antisense oligonucleotide
ggtgtgtgcctccattctcc

SEQ ID NO: 28 - DNA, 20 bp, Artificial
  antisense oligonucleotide
cccttcttccttcctctgcc

SEQ ID NO: 29 - DNA, 20 bp, Artificial
  antisense oligonucleotide
gcaagtcctcctcagcctga

SEQ ID NO: 30 - DNA, 20 bp, Artificial
  antisense oligonucleotide
ggcctgagcaagtcctcctc

SEQ ID NO: 31 - DNA, 20 bp, Artificial
  antisense oligonucleotide
gctgggtcctgtccaaaggc

SEQ ID NO: 32 - DNA, 20 bp, Artificial
  antisense oligonucleotide
gttggttgtttccctaccca

SEQ ID NO: 33 - DNA, 20 bp, Artificial
  antisense oligonucleotide
gtctcccccatgtactggac

SEQ ID NO: 34 - DNA, 20 bp, Artificial
  antisense oligonucleotide
gggaagccctgctcagtgga

SEQ ID NO: 35 - DNA, 20 bp, Artificial
  antisense oligonucleotide
taccctcacccacctctgtg

SEQ ID NO: 36 - DNA, 20 bp, Artificial
  antisense oligonucleotide
gctccatctctacctcaggt

SEQ ID NO: 37 - DNA, 20 bp, Artificial
  antisense oligonucleotide
gctcccccagaagccacact

SEQ ID NO: 38 - DNA, 20 bp, Artificial
  antisense oligonucleotide
ccccctctagtgtatggttc

SEQ ID NO: 39 - DNA, 20 bp, Artificial
  antisense oligonucleotide
tcctagcagttagcagggtc

SEQ ID NO: 40 - DNA, 20 bp, Artificial
  antisense oligonucleotide
gtctgtctgtctggtttggg

SEQ ID NO: 41 - DNA, 20 bp, Artificial
  antisense oligonucleotide
ggtggtttcttgagcttgct

SEQ ID NO: 42 - DNA, 20 bp, Artificial
  antisense oligonucleotide
gtttggcttgggcttgctgt

SEQ ID NO: 43 - DNA, 20 bp, Artificial
  antisense oligonucleotide
tgaagagagttttccctctg

SEQ ID NO: 44 - DNA, 20 bp, Artificial
  antisense oligonucleotide
gccatctatgcagaaacctc

SEQ ID NO: 45 - DNA, 20 bp, Artificial
  antisense oligonucleotide
gcctttggtgccattttcaa

SEQ ID NO: 46 - DNA, 20 bp, Artificial
  antisense oligonucleotide
cagcctgtgcctcttcctgt

SEQ ID NO: 47 - DNA, 20 bp, Artificial
  antisense oligonucleotide
gccttccttctcatgccatc

SEQ ID NO: 48 - DNA, 20 bp, Artificial
  antisense oligonucleotide
gaagatgctgctcttgctgt

SEQ ID NO: 49 - DNA, 20 bp, Artificial
  antisense oligonucleotide
atttccacctcagttgtagc

SEQ ID NO: 50 - DNA, 20 bp, Artificial
  antisense oligonucleotide
tggtgctccattgccataaa

SEQ ID NO: 51 - DNA, 20 bp, Artificial
  antisense oligonucleotide
tgccacttccatgcagtccc

SEQ ID NO: 52 - DNA, 20 bp, Artificial
  antisense oligonucleotide
tgagcaacatctccaggatg

SEQ ID NO: 53 - DNA, 20 bp, Artificial
  antisense oligonucleotide
gccataggctgtccacttga

SEQ ID NO: 54 - DNA, 20 bp, Artificial
  antisense oligonucleotide
atgagacacaccagcagcac

SEQ ID NO: 55 - DNA, 20 bp, Artificial
  antisense oligonucleotide
agaagggccaggtatcctgc

SEQ ID NO: 56 - DNA, 20 bp, Artificial
  antisense oligonucleotide
catgaagatgtcctgacctc

SEQ ID NO: 57 - DNA, 20 bp, Artificial
  antisense oligonucleotide
gcttctttgagcccagcttt

SEQ ID NO: 58 - DNA, 20 bp, Artificial
  antisense oligonucleotide
gccaggaacattcccacaat

SEQ ID NO: 59 - DNA, 20 bp, Artificial
  antisense oligonucleotide
gcagcaggccatcccaacca

SEQ ID NO: 60 - DNA, 20 bp, Artificial
  antisense oligonucleotide
gcactttcctctgtggctac

SEQ ID NO: 61 - DNA, 20 bp, Artificial
  antisense oligonucleotide
gctcaccattggcagatcca

SEQ ID NO: 62 - DNA, 20 bp, Artificial
  antisense oligonucleotide
gtgtgtgcctccattctcca

SEQ ID NO: 63 - DNA, 20 bp, Artificial
  antisense oligonucleotide
cttggattctctgacctctt

SEQ ID NO: 64 - DNA, 20 bp, Artificial
  antisense oligonucleotide
cagtaaccacctgcccaagc

SEQ ID NO: 65 - DNA, 20 bp, Artificial
  antisense oligonucleotide
gaagcccatgtggagaagat

SEQ ID NO: 66 - DNA, 20 bp, Artificial
  antisense oligonucleotide
tggttgtttccctacccaga

SEQ ID NO: 67 - DNA, 20 bp, Artificial
  antisense oligonucleotide
gccctaggatttggttttac

SEQ ID NO: 68 - DNA, 20 bp, Artificial
  antisense oligonucleotide
ccctagagataactggtgct

SEQ ID NO: 69 - DNA, 20 bp, Artificial
  antisense oligonucleotide
ggctccatctctacctcagg

SEQ ID NO: 70 - DNA, 20 bp, Artificial
  antisense oligonucleotide
ctgcagcatcttggctttga

SEQ ID NO: 71 - DNA, 20 bp, Artificial
  antisense oligonucleotide
aagtgcttctactgcaaggc

SEQ ID NO: 72 - DNA, 20 bp, Artificial
  antisense oligonucleotide
cctagcagttagcagggtca

SEQ ID NO: 73 - DNA, 20 bp, Artificial
  antisense oligonucleotide
gccagaagccatttcctatt

SEQ ID NO: 74 - DNA, 20 bp, Artificial
  antisense oligonucleotide
gcccatctcaagacctaaga

SEQ ID NO: 75 - DNA, 20 bp, Artificial
  antisense oligonucleotide
gtgctacaggtaagccaggc

SEQ ID NO: 76 - DNA, 20 bp, Artificial
  antisense oligonucleotide
tgctatcagacagaggctgc

SEQ ID NO: 77 - DNA, 20 bp, Artificial
  antisense oligonucleotide
ctggcagagggaacatggct

SEQ ID NO: 78 - DNA, 20 bp, Artificial
  antisense oligonucleotide
acagaggttccagagtgtta

SEQ ID NO: 79 - DNA, 20 bp, Artificial
  antisense oligonucleotide
agctatattccctctaccaa

SEQ ID NO: 80 - DNA, 20 bp, Artificial
  antisense oligonucleotide
ctggtctttggtttgaggaa

SEQ ID NO: 81 - DNA, 20 bp, Artificial
  antisense oligonucleotide
gggactgggctgagaacaac

SEQ ID NO: 82 - DNA, 20 bp, Artificial
  antisense oligonucleotide
actctgttatatacctgcag

SEQ ID NO: 83 - DNA, 20 bp, Artificial
  antisense oligonucleotide
ttaatgtgatgaggcagctt

SEQ ID NO: 84 - DNA, 20 bp, Artificial
  antisense oligonucleotide
ctggaaggaaaggtggttga

SEQ ID NO: 85 - DNA, 20 bp, Artificial
  antisense oligonucleotide
agtttcttctaccctccccc

SEQ ID NO: 86 - DNA, 20 bp, Artificial
  antisense oligonucleotide
gcactctccccctttacccc

SEQ ID NO: 87 - DNA, 20 bp, Artificial
  antisense oligonucleotide
gccctccctctgtctatccc

SEQ ID NO: 88 - DNA, 20 bp, Artificial
  antisense oligonucleotide
ggttctgtgccctgtccagc

SEQ ID NO: 89 - DNA, 20 bp, Artificial
  antisense oligonucleotide
gtgtgtttcctgctctctcc

SEQ ID NO: 90 - DNA, 20 bp, Artificial
  antisense oligonucleotide
cctgccagccttgttcctct

SEQ ID NO: 91 - DNA, 20 bp, Artificial
  antisense oligonucleotide
gggtgtctcctcccaggacc

SEQ ID NO: 92 - DNA, 20 bp, Artificial
  antisense oligonucleotide
cccacctcttagccctctcc

SEQ ID NO: 93 - DNA, 20 bp, Artificial
  antisense oligonucleotide
agcactgggctggctctcct

SEQ ID NO: 94 - DNA, 20 bp, Artificial
  antisense oligonucleotide
gctctccctcctaccaggtc

SEQ ID NO: 95 - DNA, 20 bp, Artificial
  antisense oligonucleotide
tcctccccagaggccagttt

SEQ ID NO: 96 - DNA, 20 bp, Artificial
  antisense oligonucleotide
agccctcagtccaccaggca

SEQ ID NO: 97 - DNA, 20 bp, Artificial
  antisense oligonucleotide
tcccccatctccctaagcct

SEQ ID NO: 98 - DNA, 20 bp, Artificial
  antisense oligonucleotide
gtagctgccacccttcctgc

SEQ ID NO: 99 - DNA, 20 bp, Artificial
  antisense oligonucleotide
tttgccccacctccctccct

SEQ ID NO: 100 - DNA, 20 bp, Artificial
  antisense oligonucleotide
gcctctgcttgccttctccc

SEQ ID NO: 101 - DNA, 20 bp, Artificial
  antisense oligonucleotide
ggctgctactcctccccaca

SEQ ID NO: 102 - DNA, 20 bp, Artificial
  antisense oligonucleotide
gcctccctcttagccactgg

SEQ ID NO: 103 - DNA, 20 bp, Artificial
  antisense oligonucleotide
gcaggccctcttccctaagt

SEQ ID NO: 104 - DNA, 20 bp, Artificial
  antisense oligonucleotide
ggttgctctttccctgtggc

SEQ ID NO: 105 - DNA, 20 bp, Artificial
  antisense oligonucleotide
gccctgcagcaccagcactc

SEQ ID NO: 106 - DNA, 20 bp, Artificial
  antisense oligonucleotide
ctccccttctcccacagctt

SEQ ID NO: 107 - DNA, 20 bp, Artificial
  antisense oligonucleotide
tccccaccctctttagtccc

SEQ ID NO: 108 - DNA, 20 bp, Artificial
  antisense oligonucleotide
ggcccagtctcactttggct

SEQ ID NO: 109 - DNA, 20 bp, Artificial
  antisense oligonucleotide
agtcccctgggtcagcttca

SEQ ID NO: 110 - DNA, 20 bp, Artificial
  antisense oligonucleotide
ccacacaagctgccccctcc

SEQ ID NO: 111 - DNA, 20 bp, Artificial
  antisense oligonucleotide
ccccacctggctgatgcccc

SEQ ID NO: 112 - DNA, 20 bp, Artificial
  antisense oligonucleotide
cctctggtttgctctcctgc

SEQ ID NO: 113 - DNA, 20 bp, Artificial
  antisense oligonucleotide
agtcaggtcccctttcccct

SEQ ID NO: 114 - DNA, 20 bp, Artificial
  antisense oligonucleotide
tccttctgcctgcctgccag

SEQ ID NO: 115 - DNA, 20 bp, Artificial
  antisense oligonucleotide
tggtggtttcttgagcttgc

SEQ ID NO: 116 - DNA, 20 bp, Artificial
  antisense oligonucleotide
ggcctttggtggtttcttga

SEQ ID NO: 117 - DNA, 20 bp, Artificial
  antisense oligonucleotide
tgtttggcttgggcttgctg

SEQ ID NO: 118 - DNA, 20 bp, Artificial
  antisense oligonucleotide
ccatctatgcagaaacctct

SEQ ID NO: 119 - DNA, 20 bp, Artificial
  antisense oligonucleotide
ggtgccattttcaagatagc

SEQ ID NO: 120 - DNA, 20 bp, Artificial
  antisense oligonucleotide
catacactggtatccctctg

SEQ ID NO: 121 - DNA, 20 bp, Artificial
  antisense oligonucleotide
tcatacactggtatccctct

SEQ ID NO: 122 - DNA, 20 bp, Artificial
  antisense oligonucleotide
ttcatacactggtatccctc

SEQ ID NO: 123 - DNA, 20 bp, Artificial
  antisense oligonucleotide
ctttcatacactggtatccc

SEQ ID NO: 124 - DNA, 20 bp, Artificial
  antisense oligonucleotide
gctttcatacactggtatcc

SEQ ID NO: 125 - DNA, 20 bp, Artificial
  antisense oligonucleotide
ggttccttcctgctttcata

SEQ ID NO: 126 - DNA, 20 bp, Artificial
  antisense oligonucleotide
gcttcttaagttgctccaac

SEQ ID NO: 127 - DNA, 20 bp, Artificial
  antisense oligonucleotide
accttcttcctctatggcat

SEQ ID NO: 128 - DNA, 20 bp, Artificial
  antisense oligonucleotide
caccttcttcctctatggca

SEQ ID NO: 129 - DNA, 20 bp, Artificial
  antisense oligonucleotide
tgcactctttgagctgagtt

SEQ ID NO: 130 - DNA, 20 bp, Artificial
  antisense oligonucleotide
ctccctattctgttgtctgg

SEQ ID NO: 131 - DNA, 20 bp, Artificial
  antisense oligonucleotide
tttccacctcagttgtagcc

SEQ ID NO: 132 - DNA, 20 bp, Artificial
  antisense oligonucleotide
aggaggctaattggtccatg

SEQ ID NO: 133 - DNA, 20 bp, Artificial
  antisense oligonucleotide
taggaggctaattggtccat

SEQ ID NO: 134 - DNA, 20 bp, Artificial
  antisense oligonucleotide
ctgctagactcagttccatt

SEQ ID NO: 135 - DNA, 20 bp, Artificial
  antisense oligonucleotide
gagttcacagtcctggttga

SEQ ID NO: 136 - DNA, 20 bp, Artificial
  antisense oligonucleotide
cagcaccaccaagttgccaa

SEQ ID NO: 137 - DNA, 20 bp, Artificial
  antisense oligonucleotide
ttcagcaccaccaagttgcc

SEQ ID NO: 138 - DNA, 20 bp, Artificial
  antisense oligonucleotide
ggccacacccttcttgatac

SEQ ID NO: 139 - DNA, 20 bp, Artificial
  antisense oligonucleotide
aggccacacccttcttgata

SEQ ID NO: 140 - DNA, 20 bp, Artificial
  antisense oligonucleotide
caggccacacccttcttgat

SEQ ID NO: 141 - DNA, 20 bp, Artificial
  antisense oligonucleotide
ccaggccacacccttcttga

SEQ ID NO: 142 - DNA, 20 bp, Artificial
  antisense oligonucleotide
tgcacctttagtttggtcca

SEQ ID NO: 143 - DNA, 20 bp, Artificial
  antisense oligonucleotide
gccattgccattcttctgga

SEQ ID NO: 144 - DNA, 20 bp, Artificial
  antisense oligonucleotide
ggagctggtgtcatctagtt

SEQ ID NO: 145 - DNA, 20 bp, Artificial
  antisense oligonucleotide
cacagggacctcttctactt

SEQ ID NO: 146 - DNA, 20 bp, Artificial
  antisense oligonucleotide
ttccacagggacctcttcta

SEQ ID NO: 147 - DNA, 20 bp, Artificial
  antisense oligonucleotide
ggccttccttctcatgccat

SEQ ID NO: 148 - DNA, 20 bp, Artificial
  antisense oligonucleotide
gcgagcctgggatgctgagc

SEQ ID NO: 149 - DNA, 20 bp, Artificial
  antisense oligonucleotide
tacttctccacgctgctgcc

SEQ ID NO: 150 - DNA, 20 bp, Artificial
  antisense oligonucleotide
gtgtctgtctccaccatcat

SEQ ID NO: 151 - DNA, 20 bp, Artificial
  antisense oligonucleotide
gtgcctccattctccagctt

SEQ ID NO: 152 - DNA, 20 bp, Artificial
  antisense oligonucleotide
ccttcctctgcccgctgctg

SEQ ID NO: 153 - DNA, 20 bp, Artificial
  antisense oligonucleotide
gcaagtcctcctcagcctga

SEQ ID NO: 154 - DNA, 20 bp, Artificial
  antisense oligonucleotide
gcctgagcaagtcctcctca

SEQ ID NO: 155 - DNA, 20 bp, Artificial
  antisense oligonucleotide
tcgacctgctgtcctggtga

SEQ ID NO: 156 - DNA, 20 bp, Artificial
  antisense oligonucleotide
ttctcgacctgctgtcctgg